(19) United States
(12) Patent Application Publication
(10) Pub. No.: US 2021/0180016 A1
(43) Pub. Date: Jun. 17, 2021

(54) PLATELETS LOADED WITH mRNA

(71) Applicant: Cellphire, Inc., Rockville, MD (US)

(72) Inventors: Keith Andrew Moskowitz, Westfield, IN (US); Amber Nicole Lee, Rockville, MD (US)

(21) Appl. No.: 17/104,528

(22) Filed: Nov. 25, 2020

Related U.S. Application Data
(60) Provisional application No. 62/940,995, filed on Nov. 27, 2019.

(51) Int. Cl. C12N 5/078; C12N 15/113

(52) U.S. Cl. CPC C12N 5/0644; C12N 2320/32; C12N 15/113

(57) ABSTRACT

Provided herein are mRNA agent-loaded platelets, methods of preparing mRNA agent-loaded platelets, and methods of using mRNA agent-loaded platelets. In some embodiments, methods of loading mRNA agents into platelets include contacting platelets with an mRNA agent, a transfection reagent, and a loading buffer that can include a salt, a base, a loading agent, and optionally at least one organic solvent.

### CROSS-REFERENCE TO RELATED APPLICATIONS

This application claims priority to U.S. Provisional Patent Application No. 62/940,995, filed on Nov. 27, 2019, the contents of which are incorporated herein by reference in its entirety.

### TECHNICAL FIELD

Provided herein are compositions and methods for use of platelets, platelet derivatives, or thrombosomes (e.g., freeze-dried platelet derivatives) as biological carriers of cargo, such as mRNA agents, also referred to herein as mRNA agent-loaded platelets, platelet derivatives, or thrombosomes. Also provided herein are methods of preparing platelets, platelet derivatives, or thrombosomes loaded with the mRNA agent of interest.

mRNA agent-loaded platelets described herein can be stored under typical ambient conditions, refrigerated, cryopreserved, for example with dimethyl sulfoxide (DMSO), and/or lyophilized after stabilization (e.g., to form thrombosomes)

### BACKGROUND

Blood is a complex mixture of numerous components. In general, blood can be described as comprising four main parts: red blood cells, white blood cells, platelets, and plasma. The first three are cellular or cell-like components, whereas the fourth (plasma) is a liquid component comprising a wide and variable mixture of salts, proteins, and other factors necessary for numerous bodily functions. The components of blood can be separated from each other by various methods. In general, differential centrifugation is most commonly used currently to separate the different components of blood based on size and, in some applications, density.

Unactivated platelets, which are also commonly referred to as thrombocytes, are small, often irregularly-shaped (e.g., discoidal or ovoidal) megakaryocyte-derived components of blood that are involved in the clotting process. They aid in protecting the body from excessive blood loss due not only to trauma or injury, but to normal physiological activity as well. Megakaryocytes are also known to specifically transfer, rather than randomly transfer, mRNAs to platelets during thrombopoiesis (Rowley, J. W., et. al., Platelet mRNA: the meaning behind the message, Curr Opin Hematol, 19(5): 385-91, doi 10.1097/MOH.0b013e328357010e (2012)). Additionally, while platelets are anucleate and do not contain DNA, they do contain mRNAs, translational machinery, intact spliceosomes (e.g., capable of synthesizing protein) and functional transcription factors (Lannan, K. L., et. al., Breaking the Mold: Transcription Factors in the Anuceleate Platelet and Platelet-Derived Microparticles, Front Imunnol., 6:48, doi: 10.3389/fimmu.2015.00048 (2015)). Platelets are considered crucial in normal hemostasis, providing the first line of defense against blood escaping from injured blood vessels. Platelets generally function by adhering to the lining of broken blood vessels, in the process becoming activated, changing to an amorphous shape, and interacting with components of the clotting system that are present in plasma or are released by the platelets themselves or other components of the blood. Purified platelets have found use in treating subjects with low platelet count (thrombocytopenia) and abnormal platelet function (thrombasthenia). Concentrated platelets are often used to control bleeding after injury or during acquired platelet function defects or deficiencies, for example those occurring during surgery and those due to the presence of platelet inhibitors.

Loading platelets with mRNA agents may allow targeted delivery of the mRNA agents to sites of interest. Further, mRNA agent-loaded platelets may be lyophilized or cryopreserved to allow for long-term storage. More specifically, platelets can translate loaded mRNA agents (e.g., mRNA) into their respective proteins. Thus, mRNA agent-loaded platelets can be lyophilized or cryopreserved and when rehydrated can translate the loaded mRNA agents into their respective proteins. In some embodiments, the loading of a mRNA agent in the platelets mitigates systemic side effects associated with the mRNA agent and lowers the threshold of therapeutic dose necessary by facilitating targeted treatment at site of interest. mRNA agent-loaded platelets are generally described in (Novakowski, S., et. al., Delivery of mRNA to platelets using lipid nanoparticles, Scientific Reports, 9, 552 (2019), which is incorporated herein by reference).

### SUMMARY OF THE INVENTION

In some embodiments, provided herein are methods of preparing mRNA agent-loaded platelets, mRNA agent-loaded platelet derivatives, or mRNA agent-loaded thrombosomes (e.g., freeze-dried platelet derivatives), comprising: contacting platelets, platelet derivatives, or thrombosomes with a mRNA agent, a cationic transfection reagent and at least one loading agent and optionally one or more plasticizers such as organic solvents, such as organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form the mRNA agent-loaded platelets, mRNA agent-loaded platelet derivatives, or mRNA agent-loaded thrombosomes.

In some embodiments, the methods of preparing mRNA agent-loaded platelets can include contacting the platelets, the platelet derivatives, and/or the thrombosomes with the mRNA agent and with one loading agent. In some embodiments, the methods of preparing mRNA agent-loaded platelets, mRNA agent-loaded platelet derivatives, or mRNA agent-loaded thrombosomes can include contacting the platelets, the platelet derivatives, or the thrombosomes with the mRNA agent and with multiple loading agents.

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents include, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof. The presence of organic solvents, such as ethanol, can be beneficial in the processing of platelets, platelet derivatives, and/or thrombosomes. In some embodiments, the organic solvent may open up and/or increase the flexibility of the plasma membrane of the platelets, platelet derivatives, and/or thrombosomes.

In some embodiments, provided herein is a method of preparing mRNA agent-loaded platelets, mRNA agent-loaded platelet derivatives, or mRNA agent-loaded thrombosomes, comprising: contacting platelets, platelet derivatives, or thrombosomes with a mRNA agent, a cationic transfection reagent and a loading buffer comprising a base, a loading agent, and optionally at least one organic solvent such as an organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, to form the mRNA agent-loaded platelets, the mRNA agent-loaded platelet derivatives, or the mRNA agent-loaded thrombosomes.

In some embodiments, provided herein is a method of preparing mRNA agent-loaded platelets, mRNA agent-loaded platelet derivatives, or mRNA agent-loaded thrombosomes, comprising: contacting platelets, platelet derivatives, or thrombosomes with a mRNA agent, a cationic transfection reagent and a loading buffer comprising a salt, a base, a loading agent, and optionally at least one organic solvent to form the mRNA agent-loaded platelets, mRNA agent-loaded platelet derivatives, or the mRNA agent-loaded thrombosomes.

In some embodiments, provided herein is a method of preparing mRNA agent-loaded platelets, mRNA agent-loaded platelet derivatives, or mRNA agent-loaded thrombosomes, comprising: contacting platelets, platelet derivatives, or thrombosomes with a mRNA agent and with a loading agent and optionally at least one organic solvent to form the mRNA agent-loaded platelets, the mRNA agent-loaded platelet derivatives, or the mRNA agent-loaded thrombosomes.

In some embodiments, provided herein is a method of preparing mRNA agent-loaded platelets, mRNA agent-loaded platelet derivatives, or mRNA agent-loaded thrombosomes, comprising: contacting platelets, platelet derivatives, or thrombosomes with a mRNA agent, a cationic transfection reagent, and a loading buffer comprising a base, a loading agent, and optionally at least one organic solvent to form the mRNA agent-loaded platelets, the mRNA agent-loaded platelet derivatives, or the mRNA agent-loaded thrombosomes.

In some embodiments, provided herein is a method of preparing mRNA agent-loaded platelets, mRNA agent-loaded platelet derivatives, or mRNA agent-loaded thrombosomes, comprising: contacting platelets, platelet derivatives, or thrombosomes with a mRNA agent, a cationic transfection reagent, and a loading buffer comprising a salt, a base, a loading agent, and optionally at least one organic solvent to form the mRNA agent-loaded platelets, the mRNA agent-loaded platelet derivatives, or the mRNA agent-loaded thrombosomes.

In some embodiments, provided herein is a method of preparing mRNA agent-loaded platelets, mRNA agent-loaded platelet derivatives, or mRNA agent-loaded thrombosomes, comprising: (a) providing platelets, platelet derivatives, or thrombosomes; and (b) contacting the platelets, the platelet derivatives, or the thrombosomes with a mRNA agent, a cationic transfection reagent, and a loading buffer comprising a salt, a base, a loading agent, and optionally at least one organic solvent to form the mRNA agent-loaded platelets, mRNA agent-loaded platelet derivatives, or the mRNA agent-loaded thrombosomes. In some embodiments, the methods further include cryopreserving the mRNA agent-loaded platelets, mRNA agent-loaded platelet derivatives, or the mRNA agent-loaded thrombosomes. In some embodiments, the methods further include cold storing the mRNA agent-loaded platelets, mRNA agent-loaded platelet derivatives, or the mRNA agent-loaded thrombosomes. In some embodiments, the methods further include drying the mRNA agent-loaded platelets or the mRNA agent-loaded platelet derivatives. In some embodiments, the methods further include freeze-drying the mRNA agent-loaded platelets or the mRNA agent-loaded platelet derivatives. In such embodiments, the methods may further include rehydrating the mRNA agent-loaded platelets, mRNA agent-loaded platelet derivatives, or mRNA agent-loaded thrombosomes obtained from the drying step. In some embodiments, the methods that further include drying the mRNA agent-loaded platelets or mRNA agent-loaded platelet derivatives and rehydrating the mRNA agent-loaded platelets or the mRNA agent-loaded platelet derivatives obtained from the drying step provides rehydrated platelets or platelet derivatives comprising at least 10% of the amount of the mRNA agent of step (b). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or platelet derivatives comprising from about 0.1 nM to about 10 μM, such as about 1 nM to about 1 μM, such as about 10 nM to 10 μM, such as about 100 nM of the mRNA agent.

In some embodiments, the platelets, platelet derivatives, or thrombosomes are contacted with the mRNA agent and with the buffer sequentially, in either order.

In some embodiments, provided herein is a method of preparing mRNA agent-loaded platelets, mRNA agent-loaded platelet derivatives, or mRNA agent-loaded thrombosomes, comprising: (1) contacting platelets, platelet derivatives, or thrombosomes with a mRNA agent to form a first composition; and (2) contacting the first composition with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (2). In some embodiments, the first composition is contacted with a cationic transfection agent during the contacting step (2). In some embodiments, the first composition is contacted with a cationic transfection agent both prior to and during the contacting step (2). In some embodiments, the methods further include drying the mRNA agent-loaded platelets or the mRNA agent-loaded platelet derivatives obtained in step (2). In some embodiments, the methods further include cryopreserving, lyopreserving (e.g., freeze-drying) the mRNA agent-loaded platelets or the mRNA agent-loaded platelet derivatives. In some embodiments, the methods further include cold storing the mRNA agent-loaded platelets, the mRNA agent-loaded platelet derivatives, the mRNA agent-loaded thrombosomes, or compositions containing mRNA agent-loaded platelets at suitable storage temperatures, such as standard room temperature storing (e.g., storing at a temperature ranging from about 20 to about 30° C.) or cold storing (e.g., storing at a temperature ranging from about 1 to about −80° C.).

In some embodiments, the methods further include cryopreserving, freeze-drying, thawing, rehydrating, and combinations thereof, the mRNA agent loaded platelets, the mRNA agent-loaded platelet derivatives, or the mRNA agent-loaded thrombosomes. For example, in such embodiments, the methods may further include rehydrating the mRNA agent-loaded platelets, the mRNA agent-loaded platelet derivatives, or the mRNA agent-loaded thrombosomes obtained from the drying step. In some embodiments, the methods that further include drying the mRNA agent-loaded platelets or mRNA agent-loaded platelet derivatives and rehydrating the mRNA agent-loaded platelets or the mRNA agent-loaded platelet derivatives obtained from the drying step provides rehydrated platelets or platelet derivatives comprising at least 10% of the amount of the mRNA agent of step (1). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or platelet derivatives comprising from about 0.1 nM to about 10 μM, such as about 1 nM to about 1 μM, such as about 10 nM to 10 μM, such as about 100 nM of the mRNA agent of step (1).

In some embodiments provided herein is a method of preparing mRNA agent-loaded platelets, mRNA agent-loaded platelet derivatives, or mRNA agent-loaded thrombosomes, comprising: (1) contacting the platelets, platelet derivatives, or thrombosomes with a buffer comprising a salt, a base, a loading agent, and optionally ethanol, to form a first composition; and (2) contacting the first composition with a mRNA agent, to form the mRNA agent-loaded platelets, the mRNA agent-loaded platelet derivatives, or the mRNA agent-loaded thrombosomes. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (2). In some embodiments, the first composition is contacted with a cationic transfection agent during the contacting step (2). In some embodiments, the first composition is contacted with a cationic transfection agent both prior to and during the contacting step (2). In some embodiments, the methods further include drying the mRNA agent-loaded platelets, the mRNA agent-loaded platelet derivatives, or the mRNA agent-loaded thrombosomes obtained in step (2). In some embodiments, the methods further include freeze-drying the mRNA agent-loaded platelets or the mRNA agent-loaded platelet derivatives. In such embodiments, the methods may further include rehydrating the mRNA agent-loaded platelets or the mRNA agent-loaded platelet derivatives obtained from the drying step. In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or platelet derivatives comprising at least 10% of the amount of the mRNA agent of step (2). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising from about 0.1 nM to about 10 such as about 1 nM to about 1 such as about 10 nM to 10 such as about 100 nM of the mRNA agent of step (2).

In some embodiments, the platelets or thrombosomes are contacted with the mRNA agent and with the buffer concurrently.

Thus, in some embodiments provided herein is a method of preparing mRNA agent-loaded platelets, the mRNA agent-loaded platelet derivatives, or the mRNA agent-loaded thrombosomes, comprising: contacting the platelets, the platelet derivatives, or the thrombosomes with a mRNA agent and a cationic transfection reagent in the presence of a buffer comprising a salt, a base, a loading agent, and optionally ethanol, to form the mRNA agent-loaded platelets, the mRNA agent-loaded platelet derivatives, or the mRNA agent-loaded thrombosomes.

In some embodiments, the methods further include drying the mRNA agent-loaded platelets or the mRNA agent-loaded platelet derivatives. In some embodiments, the methods further include freeze-drying the mRNA agent-loaded platelets or the mRNA agent-loaded platelet derivatives. In such embodiments, the methods further include rehydrating the mRNA agent-loaded platelets or the mRNA agent-loaded platelet derivatives obtained from the drying step.

In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or the thrombosomes comprising at least 10% of the amount of the mRNA agent prior to loading.

In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising from about 0.1 nM to about 10 such as about 1 nM to about 1 such as about 10 nM to 10 such as about 100 nM of the mRNA agent.

In some embodiments of the methods of preparing cargo-loaded platelets, such as mRNA agent-loaded platelets, as provided herein, the methods do not comprise contacting platelets, platelet derivatives, or thrombosomes with ethanol.

In some embodiments of the methods of preparing cargo-loaded platelets, such as mRNA agent-loaded platelets, as provided herein, the methods do not comprise contacting platelets, platelet derivatives, or thrombosomes with a solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.

In some embodiments of the methods of preparing cargo-loaded platelets, such as mRNA agent-loaded platelets, as provided herein, the methods do not comprise contacting platelets, platelet derivatives, or thrombosomes with an organic solvent.

In some embodiments of the methods of preparing cargo-loaded platelets, such as mRNA agent-loaded platelets, as provided herein, the methods do not comprise contacting platelets, platelet derivatives, or thrombosomes with a solvent.

In some embodiments of the methods of preparing cargo-loaded platelets, such as mRNA agent-loaded platelets, as provided herein, the methods comprise contacting platelets, platelet derivatives, or thrombosomes with a solvent, such as an organic solvent, such as organic solvent selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof, such as ethanol.

In some embodiments, platelets, platelet derivatives, or thrombosomes are pooled from a plurality of donors. Such platelets, platelet derivatives, and thrombosomes pooled from a plurality of donors may be also referred herein to as pooled platelets, platelet derivatives, or thrombosomes. In some embodiments, the donors are more than 5, such as more than 10, such as more than 20, such as more than 50, such as up to about 100 donors. In some embodiments, the donors are from about 5 to about 100, such as from about 10 to about 50, such as from about 20 to about 40, such as from about 25 to about 35.

Thus, provided herein in some embodiments is a method of preparing mRNA agent-loaded platelets, mRNA agent-loaded platelet derivatives, or mRNA agent-loaded thrombosomes comprising: (A) pooling platelets, platelet derivatives, or thrombosomes from a plurality of donors; and (B) contacting the platelets, platelet derivatives, or thrombosomes from (A) with a mRNA agent, a cationic transfection reagent, and with a loading buffer comprising a salt, a base, a loading agent, and optionally ethanol, to form the mRNA agent-loaded platelets, the mRNA agent-loaded platelet derivatives, or the mRNA agent-loaded thrombosomes. In some embodiments, the methods further include drying the mRNA agent-loaded platelets or the mRNA agent-loaded platelet derivatives obtained in (B). In some embodiments, the methods further include freeze-drying the mRNA agent-loaded platelets or the mRNA agent-loaded platelet derivatives. In such embodiments, the methods may further include rehydrating the mRNA agent-loaded platelets or the mRNA agent-loaded platelet derivatives obtained from the drying step. In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or rehydrated platelet derivatives comprising at least 10% of the amount of the mRNA agent of step (B). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or rehydrated platelet derivatives comprising from about 0.1 nM to about 10 μM, such as about 1 nM to about 1 μM, such as about 10 nM to 10 μM, such as about 100 nM of the mRNA agent of step (B).

In some embodiments, the pooled platelets, platelet derivatives, or thrombosomes are contacted with the mRNA agent and with the buffer sequentially, in either order.

Thus, provided herein in some embodiments is a method of preparing mRNA agent-loaded platelets, mRNA agent-loaded platelet derivatives, or mRNA agent-loaded thrombosomes comprising: (A) pooling platelets, platelet derivatives, or thrombosomes from a plurality of donors; and (B) (1) contacting the platelets, platelet derivatives, or thrombosomes from (A) with a mRNA agent to form a first composition; and (B) (2) contacting the first composition with a buffer comprising a salt, a base, a loading agent, and optionally ethanol, to form the mRNA agent-loaded platelets, mRNA agent-loaded platelet derivatives, or mRNA agent-loaded thrombosomes. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (B) (2). In some embodiments, the first composition is contacted with a cationic transfection agent during the contacting step (B) (2). In some embodiments, the first composition is contacted with a cationic transfection agent both prior to and during the contacting step (B) (2). In some embodiments, the methods further include drying the mRNA agent-loaded platelets or the mRNA agent-loaded platelet derivatives obtained in step (B) (2). In some embodiments, the methods further include freeze-drying the mRNA agent-loaded platelets or the mRNA agent-loaded platelet derivatives. In such embodiments, the methods may further include rehydrating the mRNA agent-loaded platelets or the mRNA agent-loaded platelet derivatives obtained from the drying step. In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or rehydrated platelet derivatives comprising at least 10% of the amount of the mRNA agent of step (B) (1). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or platelet derivatives comprising from about 0.1 nM to about 10 such as about 1 nM to about 1 such as about 10 nM to 10 such as about 100 nM of the mRNA agent of step (B) (1).

Thus, provided herein in some embodiments is a method of preparing mRNA agent-loaded platelets, mRNA agent-loaded platelet derivatives, or mRNA agent-loaded thrombosomes comprising: (A) pooling platelets, platelet derivatives, or thrombosomes from a plurality of donors; and (B) (1) contacting the platelets, the platelet derivatives, or the thrombosomes from (A) with a buffer comprising a salt, a base, a loading agent, and optionally ethanol, to form a first composition; and (B) (2) contacting the first composition with a mRNA agent to form the mRNA agent-loaded platelets, the mRNA agent-loaded platelet derivatives, or the mRNA agent-loaded thrombosomes. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (B) (2). In some embodiments, the first composition is contacted with a cationic transfection agent during the contacting step (B) (2). In some embodiments, the first composition is contacted with a cationic transfection agent both prior to and during the contacting step (B) (2). In some embodiments, the methods further include drying the mRNA agent-loaded platelets or the mRNA agent-loaded platelet derivatives obtained in step (B) (2). In some embodiments, the methods further include freeze-drying the mRNA agent-loaded platelets or the mRNA agent-loaded platelet derivatives. In such embodiments, the methods may further include rehydrating the mRNA agent-loaded platelets or the mRNA agent-loaded platelet derivatives obtained from the drying step. In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising at least 10% of the amount of the mRNA agent of step (B) (2). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising from about 0.1 nM to about 10 μM, such as about 1 nM to about 1 μM, such as about 10 nM to 10 μM, such as about 100 nM of the mRNA agent of step (B) (2).

In some embodiments, the pooled platelets, platelet derivatives, or thrombosomes are contacted with the mRNA agent and with the buffer concurrently.

Thus, in some embodiments provided herein is a method of preparing mRNA agent-loaded platelets, mRNA agent-loaded platelet derivatives, or mRNA agent-loaded thrombosomes, comprising: (A) pooling platelets, platelet derivatives, or thrombosomes from a plurality of donors; and (B) contacting the platelets, the platelet derivatives, or the thrombosomes with a mRNA agent and a cationic transfection reagent in the presence of a buffer comprising a salt, a base, a loading agent, and optionally ethanol, to form the mRNA agent-loaded platelets, the mRNA agent-loaded platelet derivatives, or the mRNA agent-loaded thrombosomes. In some embodiments, the methods further include drying the mRNA agent-loaded platelets or the mRNA agent-loaded platelet derivatives obtained in step (B). In some embodiments, the methods further include freeze-drying the mRNA agent-loaded platelets or the mRNA agent-loaded platelet derivatives. In such embodiments, the methods may further include rehydrating the mRNA agent-loaded platelets or the mRNA agent-loaded platelet derivatives obtained from the drying step. In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising at least 10% of the amount of the mRNA agent of step (B). In some embodiments, the methods that further include drying the mRNA agent-loaded platelets or the mRNA agent-loaded platelet derivatives and rehydrating the mRNA agent-loaded platelets or the mRNA agent-loaded platelet derivatives obtained from the drying step provides rehydrated platelets or thrombosomes comprising from about 0.1 nM to about 10 such as about 1 nM to about 1 such as about 10 nM to 10 such as about 100 nM of the mRNA agent of step (B).

In some embodiments, the methods of preparing mRNA agent-loaded platelets, mRNA agent-loaded platelet derivatives, or mRNA agent-loaded thrombosomes that include pooling platelets, platelet derivatives, or thrombosomes from a plurality of donors include a viral inactivation step.

In some embodiments, the methods of preparing mRNA agent-loaded platelets, mRNA agent-loaded platelet derivatives, or mRNA agent-loaded thrombosomes that include pooling platelets, platelet derivatives, or thrombosomes from a plurality of donors do not include a viral inactivation step.

In some embodiments, the platelets, the platelet derivatives, or the thrombosomes are loaded with the mRNA agent in a period of time of about less than 1 minute to 48 hours, such as 5 minutes to 24 hours, such as 20 minutes to 12 hours, such as 30 minutes to 6 hours, such as 1 hour to 3 hours, such as about 2 hours. In some embodiments, platelets, platelet derivatives, or thrombosomes are loaded with the mRNA agent for a time of about 1 minute, 2 minutes, 3 minutes, 4 minutes, 5 minutes, 6 minutes, 7 minutes, 8 minutes, 9 minutes, 10 minutes, 15 minutes, 20 minutes, 25 minutes, 30 minutes, 35 minutes, 40 minutes, 45 minutes, 50 minutes, 55 minutes, 1 hour, 2 hours, 3 hours, 4 hours, 5 hours, 6 hours, 7 hours, 8 hours, 9 hours 10 hours, 11 hours, 12 hours, 13 hours, 14 hours, 15 hours, 16 hours, 17 hours, 18 hours, 19 hours, 20 hours, 21 hours, 22 hours, 23 hours, 24 hours, or longer, or any time period range thereins. In some embodiments, platelets, platelet derivatives, or thrombosomes are loaded with the mRNA agent for a time of less than one minute. In some embodiments, a concentration of mRNA agent from about 0.1 nM to about 10 such as about 1 nM to about 1 such as about 10 nM to such as about 100 nM is loaded in a period of time of about less than 1 minute to 48 hours, such as 5 minutes to 24 hours, such as 20 minutes to 12 hours, such as 30 minutes to 6 hours, such as 1 hour minutes to 3 hours, such as about 2 hours.

In some embodiments, provided herein are mRNA agent-loaded platelets, mRNA agent-loaded platelet derivatives, or mRNA agent-loaded thrombosomes prepared according to any of the variety of methods disclosed herein. In some embodiments provided herein are rehydrated platelets, platelet derivatives, or thrombosomes prepared as according to any of the variety of methods disclosed herein.

In some embodiments, mRNA agent-loaded platelets, mRNA agent-loaded platelet derivatives, or mRNA agent-loaded thrombosomes protect the mRNA agent from metabolic degradation or inactivation. mRNA agent delivery with mRNA agent-loaded platelets, mRNA agent-loaded platelet derivatives, or mRNA agent-loaded thrombosomes may therefore be advantageous in treatment of diseases such as cancer, since mRNA agent-loaded platelets, mRNA agent-loaded platelet derivatives, or mRNA agent-loaded thrombosomes facilitate targeting of cancer cells while mitigating systemic side effects. mRNA agent-loaded platelets, mRNA agent-loaded platelet derivatives, or mRNA agent-loaded thrombosomes may be used in any therapeutic setting in which expedited healing process is required or advantageous.

In some embodiments, mRNA agent-loaded platelets, mRNA agent-loaded platelet derivatives, or mRNA agent-loaded thrombosomes translate the mRNA loaded agents into their respective proteins. For example, the proteins translated in mRNA agent-loaded platelets, mRNA agent-loaded platelet derivatives, or mRNA agent-loaded thrombosomes can therefore be advantageous in treatment of diseases such as cancer, since mRNA agent-loaded platelets, mRNA agent-loaded platelet derivatives, or mRNA agent-loaded thrombosomes facilitate targeting of cancer cells while mitigating systemic side effects. As another example, proteins translated in mRNA agent-loaded platelets, mRNA agent-loaded platelet derivatives, or mRNA agent-loaded thrombosomes may be used in any therapeutic setting in which expedited healing process is required or advantageous.

Accordingly, in some embodiments, provided herein is a method of treating a disease (e.g., any of the variety of diseases disclosed herein), comprising administering any of the variety of mRNA agent-loaded platelets, mRNA agent-loaded platelet derivatives, or mRNA agent-loaded thrombosomes disclosed herein. Accordingly, in some embodiments, provided herein is a method of treating a disease (e.g., any of the variety of diseases disclosed herein), comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is, Traumatic Brain injury. In some embodiments, the disease is, ITP. In some embodiments, the disease is TTP. In some embodiments, the disease is inherited disorders. In some embodiments, the disease is heart disease. In some embodiments, the disease is kidney disease. In some embodiments, the disease is a nervous system development disease. In some embodiments, the disease is hemostasis. In some embodiments, the disease is obesity.

### DESCRIPTION OF DRAWINGS

FIGS. 1A and 1B show detection of Cy5-linked EGFP mRNA after 30 minutes of incubation in samples A, B, C, G (FIG. 1A) and samples D, E, F, and G (FIG. 1B).

FIGS. 2A and 2B show detection of Cy5-linked EGFP mRNA after 120 minutes of incubation in samples A, B, C, G (FIG. 2A) and samples D, E, F and G (FIG. 2B).

FIG. 3 is a graph showing mRNA loaded platelet concentration (plts/μl) measured at 30 minutes and 120 minutes over an incubation time of 120 minutes.

FIG. 4 is a graph showing mRNA loaded platelet forward scatter height (FSC-H) measured by flow cytometry at 30 minutes and 120 minutes over an incubation time of 120 minutes.

FIG. 5 is a graph showing mRNA loaded platelet Cy5 mean fluorescent intensity measured at 30 minutes and 120 minutes over an incubation time of 120 minutes.

FIG. 6 is a graph showing Cy5-H positive mRNA loaded platelets percentage measured at 30 minutes and 120 minutes over an incubation time of 120 minutes.

FIG. 7 is a flow cytometry histogram showing measurement of mRNA loaded platelet Cy5-H after 30 minutes of incubation as measured by count [10³] in samples A, E, G, I, and J.

FIG. 8 is a flow cytometry histogram showing measurement of mRNA loaded platelet Cy5-H after 120 minutes of incubation as measured by count [10³] in samples A, E, G, I, and J.

FIG. 9 is a graph showing mRNA loaded platelet concentration (plts/μl) measured at 30 minutes and 120 minutes over an incubation time of 120 minutes in samples A, E, G, I, and J.

FIG. 10 is a graph showing mRNA loaded platelet forward scatter height (FSC-H) measured by flow cytometry at 30 minutes and 120 minutes over an incubation time of 120 minutes in samples A, E, G, I, and J.

FIG. 11 is a graph showing mRNA loaded platelet Cy5-H measured at 30 minutes and 120 minutes over an incubation time of 120 minutes in samples A, E, G, I, and J.

FIG. 12 is a graph showing mRNA loaded platelet Cy5 positivity percentage measured at 30 minutes and 120 minutes over an incubation time of 120 minutes in samples A, E, G, I, and J.

FIG. 13 is a graph showing a flow cytometry histogram for sample E measured at 30 minutes and 120 minutes over an incubation time of 120 minutes.

FIG. 14 is a graph showing a flow cytometry histogram for sample G measured at 30 minutes and 120 minutes over an incubation time of 120 minutes.

FIG. 15 is a graph showing a flow cytometry histogram for sample I measured at 30 minutes and 120 minutes over an incubation time of 120 minutes.

### DETAILED DESCRIPTION

It is to be understood that the terminology used herein is for the purpose of describing particular embodiments only, and is not intended to be limiting. Further, where a range of values is disclosed, the skilled artisan will understand that all other specific values within the disclosed range are inherently disclosed by these values and the ranges they represent without the need to disclose each specific value or range herein. For example, a disclosed range of 1-10 includes 1-9, 1-5, 2-10, 3.1-6, 1, 2, 3, 4, 5, and so forth. In addition, each disclosed range includes up to 5% lower for the lower value of the range and up to 5% higher for the higher value of the range. For example, a disclosed range of 4-10 includes 3.8-10.5. This concept is captured in this document by the term “about”.

Unless defined otherwise, all technical and scientific terms used herein have the same meaning as commonly understood by one of ordinary skill in the art to which the term belongs. Although any methods and materials similar or equivalent to those described herein can be used in the practice or testing of the present disclosure, the preferred methods and materials are now described. All publications mentioned herein are incorporated herein by reference to disclose and describe the methods and/or materials in connection with which the publications are cited. The present disclosure is controlling to the extent it conflicts with any incorporated publication.

As used herein and in the appended claims, the term “platelet” can include whole platelets, fragmented platelets, platelet derivatives, or thrombosomes. Thus, for example, reference to “mRNA agent-loaded platelets” may be inclusive of mRNA agent-loaded platelets as well as mRNA agent-loaded platelet derivatives or mRNA agent-loaded thrombosomes, unless the context clearly dictates a particular form.

The term “mRNA agent-loaded platelets” also includes mRNA agent-loaded platelets as well as mRNA agent-loaded platelet derivatives or mRNA agent-loaded thrombosomes where the platelet is capable of translating, translates, or has translated the mRNA agent into its respective protein. As used herein, “thrombosomes” (sometimes also herein called “Tsomes” or “Ts”, particularly in the Examples and Figures) are platelet derivatives that have been treated (e.g., contacted) with an incubating agent (e.g., any of the incubating agents described herein) and lyopreserved (e.g., freeze-dried). In some cases, thrombosomes can be prepared from pooled platelets. Thrombosomes can have a shelf life of 2-3 years in dry form at ambient temperature and can be rehydrated with sterile water within minutes for immediate infusion.

As used herein and in the appended claims, the term “fresh platelet” can include day of use platelets.

As used herein and in the appended claims the term “stored platelet” can include platelets stored for approximately 24 hours or longer before use.

As used herein and in the appended claims, the singular forms “a”, “an”, and “the” include plural referents unless the context clearly dictates otherwise. Thus, for example, reference to “a platelet” includes a plurality of such platelets. Furthermore, the use of terms that can be described using equivalent terms include the use of those equivalent terms. Thus, for example, the use of the term “subject” is to be understood to include the terms “patient”, “individual” and other terms used in the art to indicate one who is subject to a treatment.

In some embodiments, rehydrating the mRNA agent-loaded platelets includes adding to the platelets an aqueous liquid. In some embodiments, the aqueous liquid is water. In some embodiments, the aqueous liquid is an aqueous solution. In some embodiments, the aqueous liquid is a saline solution. In some embodiments, the aqueous liquid is a suspension.

In some embodiments, the rehydrated platelets have coagulation factor levels showing all individual factors (e.g., Factors VII, VIII and IX) associated with blood clotting at 40 international units (IU) or greater.

In some embodiments, the dried platelets, such as freeze-dried platelets, have less than about 10%, such as less than about 8%, such as less than about 6%, such as less than about 4%, such as less than about 2%, such as less than about 0.5% crosslinking of platelet membranes via proteins and/or lipids present on the membranes. In some embodiments, the rehydrated platelets, have less than about 10%, such as less than about 8%, such as less than about 6%, such as less than about 4%, such as less than about 2%, such as less than about 0.5% crosslinking of platelet membranes via proteins and/or lipids present on the membranes.

In some embodiments, the mRNA agent-loaded platelets and the dried platelets, such as freeze-dried platelets, having a particle size (e.g., diameter, max dimension) of at least about 0.2 μm (e.g., at least about 0.3 μm, at least about 0.4 μm, at least about 0.5 μm, at least about 0.6 μm, at least about 0.7 μm, at least about 0.8 μm, at least about 0.9 μm, at least about 1.0 μm, at least about 1.0 μm, at least about 1.5 μm, at least about 2.0 μm, at least about 2.5 μm, or at least about 5.0 μm). In some embodiments, the particle size is less than about 5.0 μm (e.g., less than about 2.5 μm, less than about 2.0 μm, less than about 1.5 μm, less than about 1.0 μm, less than about 0.9 μm, less than about 0.8 μm, less than about 0.7 μm, less than about 0.6 μm, less than about 0.5 μm, less than about 0.4 μm, or less than about 0.3 μm). In some embodiments, the particle size is from about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, at least 50% (e.g., at least about 55%, at least about 60%, at least about 65%, at least about 70%, at least about 75%, at least about 80%, at least about 85%, at least about 90%, at least about 95%, or at least about 99%) of platelets and/or the dried platelets, such as freeze-dried platelets, have a particle size in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, at most 99% (e.g., at most about 95%, at most about 80%, at most about 75%, at most about 70%, at most about 65%, at most about 60%, at most about 55%, or at most about 50%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm). In some embodiments, about 50% to about 99% (e.g., about 55% to about 95%, about 60% to about 90%, about 65% to about 85, about 70% to about 80%) of platelets and/or the dried platelets, such as freeze-dried platelets, are in the range of about 0.3 μm to about 5.0 μm (e.g., from about 0.4 μm to about 4.0 μm, from about 0.5 μm to about 2.5 μm, from about 0.6 μm to about 2.0 μm, from about 0.7 μm to about 1.0 μm, from about 0.5 μm to about 0.9 μm, or from about 0.6 μm to about 0.8 μm).

In some embodiments, platelets are isolated prior to treating (e.g., contacting) the platelets with a mRNA agent.

Accordingly, in some embodiments, the methods for preparing mRNA agent-loaded platelets includes: step (a) isolating platelets, for example in a liquid medium; and step (b) treating the platelets with a mRNA agent, a cationic transfection reagent, and with a loading buffer comprising a salt, a base, a loading agent, and optionally ethanol, to form the mRNA agent-loaded platelets.

Accordingly, in some embodiments, the methods for preparing mRNA agent-loaded platelets includes: step (a) isolating platelets, for example in a liquid medium; and step (b) contacting the platelets with a mRNA agent, a cationic transfection reagent, and with a loading buffer comprising a salt, a base, a loading agent, and optionally ethanol, to form the mRNA agent-loaded platelets,

Accordingly, in some embodiments, the methods for preparing mRNA agent-loaded platelets includes: step (a) isolating platelets, for example in a liquid medium; step (b) treating the platelets with a mRNA agent to form a first composition; and step (c) treating the first composition with a buffer comprising a salt, a base, a loading agent, and optionally at least one organic solvent to form the mRNA agent-loaded platelets. In some embodiments, the methods further include treating the first composition with a cationic transfection reagent to form a second composition. In some embodiments, the second composition is treated with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is treated with a cationic transfection agent prior to the treating step (c). In some embodiments, the first composition is treated with a cationic transfection agent during the treating step (c). In some embodiments, the first composition is treated with a cationic transfection agent both prior to and during the treating step (c).

Accordingly, in some embodiments, the methods for preparing mRNA agent-loaded platelets includes: step (a) isolating platelets, for example in a liquid medium; step (b) contacting the platelets with a mRNA agent to form a first composition; and step (c) contacting the first composition with a buffer comprising a salt, a base, a loading agent, and optionally at least one organic solvent to form the mRNA agent-loaded platelets. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (c). In some embodiments, the first composition is contacted with a cationic transfection agent during the contacting step (c). In some embodiments, the first composition is contacted with a cationic transfection agent both prior to and during the contacting step (c).

In some embodiments, suitable organic solvents include, but are not limited to alcohols, esters, ketones, ethers, halogenated solvents, hydrocarbons, nitriles, glycols, alkyl nitrates, water or mixtures thereof. In some embodiments, suitable organic solvents includes, but are not limited to methanol, ethanol, n-propanol, isopropanol, acetic acid, acetone, methyl ethyl ketone, methyl isobutyl ketone, methyl acetate, ethyl acetate, isopropyl acetate, tetrahydrofuran, isopropyl ether (IPE), tert-butyl methyl ether, dioxane (e.g., 1,4-dioxane), acetonitrile, propionitrile, methylene chloride, chloroform, toluene, anisole, cyclohexane, hexane, heptane, ethylene glycol, nitromethane, dimethylformamide, dimethyl sulfoxide, N-methyl pyrrolidone, dimethylacetamide, and combinations thereof.

Accordingly, in some embodiments, the methods for preparing mRNA agent-loaded platelets includes: step (a) isolating platelets, for example in a liquid medium; step (b) treating the platelets with a buffer comprising a salt, a base, a loading agent, and optionally at least one organic solvent, to form a first composition; and step (c) treating the first composition with a mRNA agent, to form the mRNA agent-loaded platelets. In some embodiments, the methods further include treating the first composition with a cationic transfection reagent to form a second composition. In some embodiments, the second composition is treated with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is treated with a cationic transfection agent prior to the treating step (c). In some embodiments, the first composition is treated with a cationic transfection agent during the treating step (c). In some embodiments, the first composition is treated with a cationic transfection agent both prior to and during the treating step (c).

Accordingly, in some embodiments, the methods for preparing mRNA agent-loaded platelets includes: step (a) isolating platelets, for example in a liquid medium; step (b) contacting the platelets with a buffer comprising a salt, a base, a loading agent, and optionally at least one organic solvent, to form a first composition; and step (c) contacting the first composition with a mRNA agent, to form the mRNA agent-loaded platelets. In some embodiments, the methods further includes contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (c). In some embodiments, the first composition is contacted with a cationic transfection agent during the contacting step (c). In some embodiments, the first composition is contacted with a cationic transfection agent both prior to and during the contacting step (c).

In some embodiments, isolating platelets includes isolating platelets from blood.

In some embodiments, platelets are donor-derived platelets. In some embodiments, platelets are obtained by a process that includes an apheresis step. In some embodiments, platelets are fresh platelets. In some embodiments, platelets are stored platelets.

In some embodiments, platelets are derived in vitro. In some embodiments, platelets are derived or prepared in a culture prior to treating the platelets with a mRNA agent. In some embodiments, preparing the platelets includes deriving or growing the platelets from a culture of megakaryocytes. In some embodiments, preparing the platelets includes deriving or growing the platelets (or megakaryocytes) from a culture of human pluripotent stem cells (PCSs), including embryonic stem cells (ESCs) and/or induced pluripotent stem cells (iPSCs).

Accordingly, in some embodiments, the methods for preparing mRNA agent-loaded platelets includes: step (a) providing platelets; and step (b) treating the platelets with a mRNA agent, a cationic transfection reagent, and with a loading buffer comprising a salt, a base, a loading agent, and optionally at least one organic solvent, to form the mRNA agent-loaded platelets.

Accordingly, in some embodiments, the methods for preparing mRNA agent-loaded platelets includes: step (a) providing platelets; and step (b) treating the platelets with a mRNA agent, a cationic transfection reagent, and with a loading buffer comprising a salt, a base, a loading agent, and optionally at least one organic solvent, to form the mRNA agent-loaded platelets.

Accordingly, in some embodiments, the methods for preparing mRNA agent-loaded platelets includes: step (a) providing platelets; step (b) treating the platelets with a mRNA agent to form a first composition; and step (c) treating the first composition with a buffer comprising a salt, a base, a loading agent, and optionally at least one organic solvent, to form the mRNA agent-loaded platelets.

Accordingly, in some embodiments, the methods for preparing mRNA agent-loaded platelets includes: step (a) providing platelets; step (b) contacting the platelets with a mRNA agent to form a first composition; and step (c) contacting the first composition with a buffer comprising a salt, a base, a loading agent, and optionally at least one organic solvent, to form the mRNA agent-loaded platelets.

Accordingly, in some embodiments, the methods for preparing mRNA agent-loaded platelets includes: step (a) providing platelets; step (b) treating the platelets with a buffer comprising a salt, a base, a loading agent, and optionally at least one organic solvent, to form a first composition; and step (c) treating the first composition with a mRNA agent, to form the mRNA agent-loaded platelets. In some embodiments, the methods further include treating the first composition with a cationic transfection reagent to form a second composition. In some embodiments, the second composition is treated with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is treated with a cationic transfection agent prior to the treating step (c). In some embodiments, the first composition is treated with a cationic transfection agent during the treating step (c). In some embodiments, the first composition is treated with a cationic transfection agent both prior to and during the treating step (c).

In some embodiments, no solvent is used. Thus, in some embodiments, the method for preparing mRNA agent-loaded platelets comprises:
a) isolating platelets, for example in a liquid medium;
   and
b) treating the platelets with an mRNA agent and with a loading buffer comprising a salt, a base, and a loading agent, to form the mRNA agent-loaded platelets, wherein the method does not comprise treating the platelets with an organic solvent such as ethanol.

Accordingly, in some embodiments, the methods for preparing mRNA agent-loaded platelets includes: step (a) providing platelets; step (b) contacting the platelets with a buffer comprising a salt, a base, a loading agent, and optionally at least one organic solvent, to form a first composition; and step (c) contacting the first composition with a mRNA agent, to form the mRNA agent-loaded platelets. In some embodiments, the methods further include contacting the first composition with a cationic transfection reagent to form a second composition. In some embodiments, the second composition is contacted with a buffer comprising a salt, a base, a loading agent, and optionally at least one organic solvent to form the mRNA agent-loaded platelets, mRNA agent-loaded platelet derivatives, or mRNA agent-loaded thrombosomes. In some embodiments, the first composition is contacted with a cationic transfection agent prior to the contacting step (c). In some embodiments, the first composition is contacted with a cationic transfection agent during the contacting step (c). In some embodiments, the first composition is contacted with a cationic transfection agent both prior to and during the contacting step (c).

In some embodiments, no solvent is used. Thus, in some embodiments, the method for preparing mRNA agent-loaded platelets comprises:

isolating platelets, for example in a liquid medium; and

contacting the platelets with an mRNA agent and with a loading buffer comprising a salt, a base, and a loading agent, to form the mRNA agent-loaded platelets, wherein the method does not comprise contacting the platelets with an organic solvent such as ethanol.

Thus, in some embodiments, the method for preparing mRNA agent-loaded platelets comprises:
a) isolating platelets, for example in a liquid medium;
b) treating the platelets with a mRNA agent to form a first composition; and
c) treating the first composition with a buffer comprising a salt, a base, and a loading agent, to form the mRNA agent-loaded platelets, wherein the method does not comprise treating the platelets with an organic solvent such as ethanol and the method does not comprise treating the first composition with an organic solvent such as ethanol.

Thus, in some embodiments, the method for preparing mRNA agent-loaded platelets comprises:

a) isolating platelets, for example in a liquid medium;

b) contacting the platelets with a mRNA agent to form a first composition; and

c) contacting the first composition with a buffer comprising a salt, a base, and a loading agent, to form the mRNA agent-loaded platelets, wherein the method does not comprise contacting the platelets with an organic solvent such as ethanol and the method does not comprise contacting the first composition with an organic solvent such as ethanol.

Thus, in some embodiments, the method for preparing mRNA agent-loaded platelets comprises: isolating platelets, for example in a liquid medium; treating the platelets with a buffer comprising a salt, a base, and a loading agent, to form a first composition; and treating the first composition with a mRNA agent, to form the mRNA agent-loaded platelets. wherein the method does not comprise treating the platelets with an organic solvent such as ethanol and the method does not comprise treating the first composition with an organic solvent such as ethanol.

Thus, in some embodiments, the method for preparing mRNA agent-loaded platelets comprises: isolating platelets, for example in a liquid medium; contacting the platelets with a buffer comprising a salt, a base, and a loading agent, to form a first composition; and contacting the first composition with a mRNA agent, to form the mRNA agent-loaded platelets. wherein the method does not comprise contacting the platelets with an organic solvent such as ethanol and the method does not comprise contacting the first composition with an organic solvent such as ethanol.

In some embodiments, the method for preparing mRNA agent-loaded platelets comprises:
a) providing platelets;
   and
b) treating the platelets with a mRNA agent-loaded and with a loading buffer comprising a salt, a base, and a loading agent, to form the mRNA agent-loaded platelets,
   wherein the method does not comprise treating the platelets with an organic solvent such as ethanol.

In some embodiments, the method for preparing mRNA agent-loaded platelets comprises:

providing platelets; and

contacting the platelets with a mRNA agent-loaded and with a loading buffer comprising a salt, a base, and a loading agent, to form the mRNA agent-loaded platelets, wherein the method does not comprise treating the platelets with an organic solvent such as ethanol.

Thus, in some embodiments, the method for preparing mRNA agent-loaded platelets comprises:
a) providing platelets;
b) treating the platelets with a mRNA agent to form a first composition; and
c) treating the first composition with a buffer comprising a salt, a base, and a loading agent, to form the mRNA agent-loaded platelets,
wherein the method does not comprise treating the platelets with an organic solvent such as ethanol and the method does not comprise treating the first composition with an organic solvent such as ethanol.

Thus, in some embodiments, the method for preparing mRNA agent-loaded platelets comprises:

a) providing platelets;

b) contacting the platelets with a mRNA agent to form a first composition; and

c) contacting the first composition with a buffer comprising a salt, a base, and a loading agent, to form the mRNA agent-loaded platelets, wherein the method does not comprise contacting the platelets with an organic solvent such as ethanol and the method does not comprise contacting the first composition with an organic solvent such as ethanol.

Thus, in some embodiments, the method for preparing mRNA agent-loaded platelets comprises:
a) providing platelets;
b) treating the platelets with a buffer comprising a salt, a base, and a loading agent, to form a first composition; and
c) treating the first composition with a mRNA agent, to form the mRNA agent-loaded platelets,
wherein the method does not comprise treating the platelets with an organic solvent such as ethanol and the method does not comprise treating the first composition with an organic solvent such as ethanol.

Thus, in some embodiments, the method for preparing mRNA agent-loaded platelets comprises:

a) providing platelets;

b) contacting the platelets with a buffer comprising a salt, a base, and a loading agent, to form a first composition; and

c) contacting the first composition with a mRNA agent, to form the mRNA agent-loaded platelets, wherein the method does not comprise contacting the platelets with an organic solvent such as ethanol and the method does not comprise contacting the first composition with an organic solvent such as ethanol.

In some embodiments, the loading agent is a saccharide. In some embodiments, the saccharide is a monosaccharide. In some embodiments, the saccharide is a disaccharide. In some embodiments, the saccharide is a non-reducing disaccharide. In some embodiments, the saccharide is sucrose, maltose, trehalose, glucose (e.g., dextrose), mannose, or xylose. In some embodiments, the loading agent is a starch.

As used herein, the term “mRNA agent” is any messenger RNA (also known as mRNA).

As used herein, the term “messenger RNA” and “mRNA” refers to a ribonucleic acid capable of being translated into protein by cellular machinery (e.g., riboprotein complexes) within a cell. Many mRNAs are naturally occurring, but mRNAs can also be synthesized by those of ordinary skill in the art, both of which can be an mRNA agent. Mature mRNAs are generally between several hundred nucleotides to several thousand nucleotides in length. However, shorter mRNAs (e.g., less than about 100 nucleotides) and longer mRNAs (e.g., about 100,000 nucleotides or more) are known in the art. Generally, mRNAs have a 5′ cap (e.g., a guanosine triphosphate nucleotide), a 3′ untranslated region, and a polyadenylated (polyA) tail.

mRNAs are distinct from other types of RNA molecules including, without limitation, micro (“miRNA”), small interfering (“siRNA”), ribosomal RNA (“rRNA”), small nuclear RNA (“snRNA”), transfer RNA (“tRNA”), and short hairpin RNA (“shRNA”). miRNA, siRNA, rRNA, snRNA, and tRNA are canonical classes of RNA molecules, the function and structure of which are well-known to those of ordinary skill in the art.

In some embodiments, the mRNA agent is an mRNA (e.g., a single species of mRNA or two or more species of mRNA).

In various methods described herein, platelets are loaded with one or more any of a variety of mRNA agents. In some embodiments, platelets are loaded with one or more mRNA. For example, any mRNA, or derivative thereof, can be loaded into platelets, such as, for example, mRNAs for the treatment of a disease (e.g., cancer), reporter mRNAs (e.g., YFP, GFP, luciferase), or any other mRNA to treat a therapeutic condition described herein.

In some embodiments, a mRNA agent (e.g., an mRNA) loaded into platelets is modified. For example, a mRNA agent can be modified to increase its stability during the platelet loading process, while the mRNA agent is loaded into the platelet, and/or after the mRNA agent's release from a platelet. In some embodiments, the modified mRNA agent's stability is increased with little or no adverse effect on its activity. For example, the modified mRNA agent can have at least 70%, 75%, 80%, 85%, 90%, 95%, 96%, 97%, 98%, 99% or more of the activity of the corresponding unmodified mRNA agent. In some embodiments, the modified mRNA agent has 100% (or more) of the activity of the corresponding unmodified mRNA agent. Various modifications that stabilize mRNA agents are known in the art. In some embodiments, the mRNA agent is stabilized by one or more of a stabilizing oligonucleotide (see, e.g., U.S. Application Publication No. 2018/0311176), a backbone/side chain modification (e.g., a 2-sugar modification such as a 2′-fluor, methoxy, or amine substitution, or a 2′-thio (—SH), 2′-azido (—N3), or 2′-hydroxymethyl (—CH2OH) modification), an unnatural nucleic acid substitution (e.g., an S-glycerol, cyclohexenyl, and/or threose nucleic acid substitution, an L-nucleic acid substitution, a locked nucleic acid (LNA) modification (e.g., the ribose moiety of an LNA nucleotide is modified with an extra bridge connecting the 2′ oxygen and 4′ carbon), conjugation with PEG, a nucleic acid bond modification or replacement (e.g., a phosphorothioate bond, a methylphosphonate bond, or a phosphorodiamidate bond), a reagent or reagents (e.g., intercalating agents such as coralyne, neomycin, and ellipticine; also see US Publication Application Nos. 2018/0312903 and 2017/0198335, each of which are incorporated herein by reference in their entireties, for further examples of stabilizing reagents).

In some embodiments, a mRNA agent (e.g., mRNA) loaded into platelets is modified to include an imaging agent. For example, a mRNA agent can be modified with an imaging agent in order to image the mRNA agent loaded platelet in vivo. In some embodiments, a mRNA agent can be modified with two or more imaging agents (e.g., any two or more of the imaging agents described herein). In some embodiments, a mRNA agent loaded into platelets is modified with a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as ⁵⁴Cu, ⁴⁸V, ⁵²Fe, ⁵⁵Co, ⁹⁴Tc or ⁶⁸Ga; or gamma-emitters such as ¹⁷¹Tc, ¹¹³In, or ⁶⁷Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to ¹²³I, ¹³¹I or ⁷⁷Br. For example, a positron-emitting radioactive non-metal can include, but is not limited to ¹¹C, ¹³N, ¹⁵O, ¹⁷F, ¹⁸F, ⁷⁵Br, ⁷⁶Br or ¹²⁴I. For example, a hyperpolarized NMR-active nucleus can include, but is not limited to ¹³C, ¹⁵N, ¹⁹F, ²⁹Si and ³¹P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bts(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals ⁶⁷Cu, ⁸⁹Sr, ⁹⁰Y, ¹⁵³Sm, ¹⁸⁵Re, ¹⁸⁸Re or ¹⁹²Ir, and non-metals ³²P, ³³P, ³⁸S, ³⁸Cl, ³⁹Cl, ⁸²Br and ⁸³Br. In some embodiments, a mRNA agent loaded into platelets can be associated with gold or other equivalent metal particles (such as nanoparticles). For example, a metal particle system can include, but is not limited to gold nanoparticles (e.g., Nanogold™).

In some embodiments, a mRNA agent loaded into platelets that is modified with an imaging agent is imaged using an imaging unit. The imaging unit can be configured to image the mRNA agent loaded platelets in vivo based on an expected property (e.g., optical property from the imaging agent) to be characterized. For example, imaging techniques (in vivo imaging using an imaging unit) that can be used, but are not limited to are: computer assisted tomography (CAT), magnetic resonance spectroscopy (MRS), magnetic resonance imaging (MRI), positron emission tomography (PET), single-photon emission computed tomography (SPECT), or bioluminescence imaging (BLI). Chen, Z., et. al., Advance of Molecular Imaging Technology and Targeted Imaging Agent in Imaging and Therapy, Biomed Res Int., 819324, doi: 10.1155/2014/819324 (2014) have described various imaging techniques and which is incorporated by reference herein in its entirety.

In some embodiments, such as embodiments wherein the platelets are treated (e.g., contacted) with the mRNA agent (e.g., mRNA) and the buffer sequentially as disclosed herein, the mRNA agent may be loaded in a liquid medium that may be modified to change the proportion of media components or to exchange components for similar products, or to add components necessary for a given application.

In some embodiments, the loading buffer and/or the liquid medium include one or more of a) water or a saline solution, b) one or more additional salts, or c) a cationic transfection agent, or d) a base. In some embodiments, the loading buffer, and/or the liquid medium, may include one or more of a) DMSO, b) one or more salts, or c) a cationic transfection agent, or d) a base.

In some embodiments, the loading agent is loaded into the platelets in the presence of an aqueous medium. In some embodiments, the loading agent is loaded in the presence of a medium comprising DMSO. As an example, one embodiment of the methods herein includes treating (e.g., contacting) platelets with a mRNA agent and with an aqueous loading buffer comprising a salt, a base, a loading agent, a cationic transfection agent, and optionally at least one organic solvent, to form the mRNA agent-loaded platelets. As an example, one embodiment of the methods herein includes treating (e.g., contacting) platelets with a mRNA agent and with a loading buffer comprising DMSO and comprising a salt, a base, a loading agent, a cationic transfection agent, and optionally ethanol, to form the mRNA agent-loaded platelets.

In some embodiments, the loading buffer and/or the liquid medium, include one or more salts selected from phosphate salts, sodium salts, potassium salts, calcium salts, magnesium salts, and any other salt that can be found in blood or blood products, or that is known to be useful in drying platelets, or any combination of two or more of these.

Preferably, these salts are present in the composition at an amount that is about the same as is found in whole blood.

In some embodiments, the mRNA agent-loaded platelets are prepared by incubating the platelets with the mRNA agent in the liquid medium for different durations at or at different temperatures from about 15-45° C., or about 22° C. In some embodiments, the mRNA agent-loaded platelets are prepared by incubating the platelets with the mRNA agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the mRNA agent-loaded platelets are prepared by incubating the platelets with the mRNA agent in the liquid medium at a temperature from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the mRNA agent-loaded platelets are prepared by incubating the platelets with the mRNA agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 200,000,000 platelets/μl.

In some embodiments, other components may include imaging agents. For example, an imaging agent can include, but is not limited to a radioactive metal ion, a paramagnetic metal ion, a gamma-emitting radioactive halogen, a positron-emitting radioactive non-metal, a hyperpolarized NMR-active nucleus, a reporter suitable for in vivo optical imaging, or a beta-emitter suitable for intravascular detection. For example, a radioactive metal ion can include, but is not limited to, positron emitters such as 54Cu, 48V, 52Fe, 55Co, 94Tc or 68Ga; or gamma-emitters such as 171Tc, 111In, 113In, or 67Ga. For example, a paramagnetic metal ion can include, but is not limited to Gd(III), a Mn(II), a Cu(II), a Cr(III), a Fe(III), a Co(II), a Er(II), a Ni(II), a Eu(III) or a Dy(III), an element comprising an Fe element, a neodymium iron oxide (NdFeO3) or a dysprosium iron oxide (DyFeO3). For example, a paramagnetic metal ion can be chelated to a polypeptide or a monocrystalline nanoparticle. For example, a gamma-emitting radioactive halogen can include, but is not limited to 123I, 131I or 77Br. For example, a positron-emitting radioactive non-metal can include, but is not limited to 11C, 13N, 15O, 17F, 18F, 75Br, 76Br or 124I. For example, a hyperpolarized NMR-active nucleus can include, but is not limited to 13C, 15N, 19F, 29Si and 31P. For example, a reporter suitable for in vivo optical imaging can include, but is not limited to any moiety capable of detection either directly or indirectly in an optical imaging procedure. For example, the reporter suitable for in vivo optical imaging can be a light scatterer (e.g., a colored or uncolored particle), a light absorber or a light emitter. For example, the reporter can be any reporter that interacts with light in the electromagnetic spectrum with wavelengths from the ultraviolet to the near infrared. For example, organic chromophoric and fluorophoric reporters include groups having an extensive delocalized electron system, e.g. cyanines, merocyanines, indocyanines, phthalocyanines, naphthalocyanines, triphenylmethines, porphyrins, pyrilium dyes, thiapyrilium dyes, squarylium dyes, croconium dyes, azulenium dyes, indoanilines, benzophenoxazinium dyes, benzothiaphenothiazinium dyes, anthraquinones, napthoquinones, indathrenes, phthaloylacridones, trisphenoquinones, azo dyes, intramolecular and intermolecular charge-transfer dyes and dye complexes, tropones, tetrazines, b/s(dithiolene) complexes, bts(benzene-dithiolate) complexes, iodoaniline dyes, b/stS.O-dithiolene) complexes. For example, the reporter can be, but is not limited to a fluorescent, a bioluminescent, or chemiluminescent polypeptide. For example, a fluorescent or chemiluminescent polypeptide is a green florescent protein (GFP), a modified GFP to have different absorption/emission properties, a luciferase, an aequorin, an obelin, a mnemiopsin, a berovin, or a phenanthridinium ester. For example, a reporter can be, but is not limited to rare earth metals (e.g., europium, samarium, terbium, or dysprosium), or fluorescent nanocrystals (e.g., quantum dots). For example, a reporter may be a chromophore that can include, but is not limited to fluorescein, sulforhodamine 101 (Texas Red), rhodamine B, rhodamine 6G, rhodamine 19, indocyanine green, Cy2, Cy3, Cy3.5, Cy5, Cy5.5, Cy7, Marina Blue, Pacific Blue, Oregon Green 88, Oregon Green 514, tetramethylrhodamine, and Alexa Fluor 350, Alexa Fluor 430, Alexa Fluor 532, Alexa Fluor 546, Alexa Fluor 555, Alexa Fluor 568, Alexa Fluor 594, Alexa Fluor 633, Alexa Fluor 647, Alexa Fluor 660, Alexa Fluor 680, Alexa Fluor 700, and Alexa Fluor 750. For example, a beta-emitter can include, but is not limited to radio metals 67Cu, 89Sr, 90Y, 153 Sm, 185Re, 188Re or 192Ir, and non-metals 32P, 33P, 38S, 38C1, 39C1, 82Br and 83Br. In some embodiments, a mRNA agent loaded into platelets can be associated with gold or other equivalent metal particles (such as nanoparticles). For example, a metal particle system can include, but is not limited to gold nanoparticles (e.g., Nanogold™)

In some embodiments, the mRNA agent-loaded platelets are prepared by incubating the platelets with the mRNA agent (e.g., mRNA) in the liquid medium for different durations. The step of incubating the platelets to load one or more mRNA agent(s) includes incubating the platelets for a time suitable for loading, as long as the time, taken in conjunction with the temperature, is sufficient for the mRNA agent to come into contact with the platelets and, preferably, be incorporated, at least to some extent, into the platelets. For example, in some embodiments, the mRNA agent-loaded platelets are prepared by incubating the platelets with the mRNA agent in the liquid medium for at least about 5 minutes (mins) (e.g., at least about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, about 42 hrs, about 48 hrs, or at least about 48 hrs. In some embodiments, the mRNA agent-loaded platelets are prepared by incubating the platelets with the mRNA agent in the liquid medium for no more than about 48 hrs (e.g., no more than about 20 mins, about 30 mins, about 1 hour (hr), about 2 hrs, about 3 hrs, about 4 hrs, about 5 hrs, about 6 hrs, about 7 hrs, about 8 hrs, about 9 hrs, about 10 hrs, about 12 hrs, about 16 hrs, about 20 hrs, about 24 hrs, about 30 hrs, about 36 hrs, or no more than about 42 hrs). In some embodiments, the mRNA agent-loaded platelets are prepared by incubating the platelets with the mRNA agent in the liquid medium from about 10 mins to about 48 hours (e.g., from about 20 mins to about 36 hrs, from about 30 mins to about 24 hrs, from about 1 hr to about 20 hrs, from about 2 hrs to about 16 hours, from about 10 mins to about 24 hours, from about 20 mins to about 12 hours, from about 30 mins to about 10 hrs, or from about 1 hr to about 6 hrs. In one embodiment, treating (e.g., contacting) platelets with an mRNA agent includes contacting the platelets with a cationic transfection reagent, and a loading buffer comprising a salt, a base, a loading agent, and optionally at least one organic solvent for a period of time, such as a period of 1 minute to 48 hours, such as 2 hours.

In some embodiments, the mRNA agent-loaded platelets are prepared by incubating the platelets with the mRNA agent in the liquid medium at different temperatures. The step of incubating the platelets to load one or more mRNA agent(s), includes incubating the platelets with the mRNA agent in the liquid medium at a temperature that, when selected in conjunction with the amount of time allotted for loading, is suitable for loading. In general, the platelets with the mRNA agent in the liquid medium are incubated at a suitable temperature (e.g., a temperature above freezing) for at least a sufficient time for the mRNA agent to come into contact with the platelets. In some embodiments, incubation is conducted at 22° C. In certain embodiments, incubation is performed at 4° C. to 45° C., such as 15° C. to 42° C. For example, in some embodiments, incubation is performed from about 18-42° C., about 20-40° C., about 22-37° C., or about 16° C., about 18° C., about 20° C., about 22° C., about 24° C., about 26° C., about 28° C., about 30° C., about 32° C., about 34° C., about 36° C., about 37° C., about 39° C., about 41° C., about 43° C., or about 45° C. for 110 to 130 (e.g., 120) minutes and for as long as 24-48 hours.

In some embodiments of the methods of preparing mRNA agent-loaded platelets disclosed herein, the methods further include acidifying the platelets, or pooled platelets, to a pH of about 6.0 to about 7.4, prior to a treating (e.g., contacting) step disclosed herein. In some embodiments, the methods include acidifying the platelets to a pH of about 6.5 to about 6.9. In some embodiments, the methods include acidifying the platelets to a pH of about 6.6 to about 6.8. In some embodiments, the acidifying includes adding to the pooled platelets a solution comprising Acid Citrate Dextrose.

In some embodiments, the platelets are isolated prior to a treating (e.g., contacting) step. In some embodiments, the methods further include isolating platelets by using centrifugation. In some embodiments, the centrifugation occurs at a relative centrifugal force (RCF) of about 800 g to about 2000 g. In some embodiments, the centrifugation occurs at relative centrifugal force (RCF) of about 1300 g to about 1800 g. In some embodiments, the centrifugation occurs at relative centrifugal force (RCF) of about 1500 g. In some embodiments, the centrifugation occurs for about 1 minute to about 60 minutes. In some embodiments, the centrifugation occurs for about 10 minutes to about 30 minutes. In some embodiments, the centrifugation occurs for about 20 minutes.

In some embodiments, the platelets are at a concentration from about 1,000 platelets/μl to about 10,000,000 platelets/μl. In some embodiments, the platelets are at a concentration from about 50,000 platelets/μl to about 4,000,000 platelets/μl. In some embodiments, the platelets are at a concentration from about 100,000 platelets/μl to about 300,000,000 platelets/μl. In some embodiments, the platelets are at a concentration from about 1,000,000 to about 2,000,000. In some embodiments, the platelets are at a concentration of about 2,000,000 platelets/μl.

In some embodiments, the buffer is a loading buffer comprising the components as listed in Table 5 herein. In some embodiments, the loading buffer includes one or more salts, such as phosphate salts, sodium salts, potassium salts, calcium salts, magnesium salts, and any other salt that can be found in blood or blood products. Exemplary salts include sodium chloride (NaCl), potassium chloride (KCl), and combinations thereof. In some embodiments, the loading buffer includes from about 0.5 mM to about 100 mM of the one or more salts. In some embodiments, the loading buffer includes from about 1 mM to about 100 mM (e.g., about 2 mM to about 90 mM, about 2 mM to about 6 mM, about 50 mM to about 100 mM, about 60 mM to about 90 mM, about 70 to about 85 mM) about of the one or more salts. In some embodiments, the loading buffer includes about 5 mM, about 75 mM, or about 80 mM of the one or more salts.

In some embodiments, the loading buffer includes one or more buffers, e.g., N-2-hydroxyethylpiperazine-N′-2-ethanesulfonic acid (HEPES), and/or sodium-bicarbonate (NaHCO₃). In some embodiments, the loading buffer includes from about 5 to about 100 mM of the one or more buffers. In some embodiments, the loading buffer includes from about 5 to about 50 mM (e.g., from about 5 mM to about 40 mM, from about 8 mM to about 30 mM, about 10 mM to about 25 mM) about of the one or more buffers. In some embodiments, the loading buffer includes about 10 mM, about 20 mM, about 25 mM, or about 30 mM of the one or more buffers.

In some embodiments, the loading buffer includes one or more saccharides, such as monosaccharides and disaccharides, including sucrose, maltose, trehalose, glucose, mannose, dextrose, and xylose. In some embodiments, the loading buffer includes from about 10 mM to about 1,000 mM of the one or more saccharides. In some embodiments, the loading buffer includes from about 50 to about 500 mM of the one or more saccharides. In embodiments, one or more saccharides is present in an amount of from 10 mM 10 to 500 mM. In some embodiments, one or more saccharides is present in an amount of from 50 mM to 200 mM. In embodiments, one or more saccharides is present in an amount from 100 mM to 150 mM.

In some embodiments, the loading buffer includes adding an organic solvent, such as ethanol, to the loading solution. In such a loading buffer, the solvent can range from about 0.1% (v/v) to about 5.0% (v/v), such as from about 0.3% (v/v) to about 3.0% (v/v), or from about 0.5% (v/v) to about 2% (v/v).

In some embodiments, the mRNA agent includes one mRNA. In some embodiments, the mRNA agent includes one or more mRNAs.

In some embodiments, the methods further include incubating the mRNA agent (e.g., mRNA) in the presence of the loading buffer prior to the treatment (e.g., contacting) step. In some embodiments, the methods further include incubating the loading buffer and a solution comprising the mRNA agent and water at about 37° C. using different incubation periods. In some embodiments, the solution includes a concentration of about 0.1 nM to about 10 μM of the mRNA agent. In some embodiments, the solution includes a concentration of about 1 nM to about 1 μM of the mRNA agent. In some embodiments, the solution includes a concentration of about 10 nM to 10 μM of the mRNA agent. In some embodiments, the solution includes a concentration of about 100 nM of the mRNA agent. In some embodiments, the incubation of the mRNA agent in the presence of the loading buffer is performed from about 1 minute to about 2 hours. In some embodiments, the incubation is performed at an incubation period of from about 5 minutes to about 1 hour. In some embodiments, the incubation is performed at an incubation period of from about 10 minutes to about 30 minutes. In some embodiments, the incubation is performed at an incubation period of about 20 minutes.

In some embodiments, the methods further include incubating the mRNA agent in the presence of a cationic transfection reagent and the loading buffer prior to the treatment step (e.g., contacting). In some embodiments, the concentration of the cationic transfection reagent is from about 0.01% v/v to about 10% v/v. In some embodiments, the concentration of the cationic transfection reagent is from about 0.5% v/v to about 8% v/v. In some embodiments, the concentration of the cationic transfection reagent is from about 1% v/v to about 5% v/v. In some embodiments, the concentration of the cationic transfection reagent is from about 2% v/v to about 3% v/v.

In some embodiments, the methods further include mixing the platelets and the complexed cationic lipid and mRNA agent (cationic lipid-mRNA agent) in the presence of the loading buffer at about room temperature (e.g., at about 20° C. to about 25° C.) using a platelet to cationic lipid-mRNA agent volume ratio of about 10:1. In some embodiments, the cationic lipid is lipofectamine. In some embodiments, the methods further include incubating the platelets and the cationic lipid-mRNA agent in the presence of the loading buffer at about temperature (e.g., about 20° C. to about 25° C.) using a platelet to cationic lipid-mRNA agent volume ratio of about 10:1, using different incubation periods.

In some embodiments, the incubation is performed at an incubation period of from about 5 minutes to about 12 hours. In some embodiments, the incubation is performed at an incubation period of from about 10 minutes to about 6 hours. In some embodiments, the incubation is performed at an incubation period of from about 15 minutes to about 3 hours. In some embodiments, the incubation is performed at an incubation period of about 2 hours. In some embodiments, the final product includes platelets and the cationic lipid-mRNA agent at a volume ratio of 10:1, with a range in volume ratio of about 1 to about 50.

In some embodiments, the concentration of mRNA agent in the mRNA agent-loaded platelets is from about 0.1 nM to about 10 M. In some embodiments, the concentration of mRNA agent in the mRNA agent-loaded platelets is from about 1 nM to about 1 M. In some embodiments, the concentration of mRNA agent in the mRNA agent-loaded platelets is from about 10 nM to 10 μM. In some embodiments, the concentration of mRNA agent in the RNA-loaded platelets is about 100 nM.

In some embodiments, the methods further include drying the mRNA agent-loaded platelets. In some embodiments, the drying step includes freeze-drying the mRNA agent-loaded platelets. In some embodiments, the methods further include rehydrating the mRNA agent-loaded platelets obtained from the drying step.

In some embodiments, mRNA agent-loaded platelets are prepared by using any of the variety of methods provided herein.

In some embodiments, rehydrated mRNA agent-loaded platelets are prepared by any one method comprising rehydrating the mRNA agent-loaded platelets provided herein.

The mRNA agent-loaded platelets may be used, for example, in therapeutic applications as disclosed herein. Additionally or alternatively, the mRNA agent-loaded platelets may be employed in functional assays. In some embodiments, the mRNA agent-loaded platelets are cold stored, cryopreserved, or lyophilized (to produce thrombosomes) prior to use in therapy or in functional assays.

Any known technique for drying platelets can be used in accordance with the present disclosure, as long as the technique can achieve a final residual moisture content of less than 5%. Preferably, the technique achieves a final residual moisture content of less than 2%, such as 1%, 0.5%, or 0.1%. Non-limiting examples of suitable techniques are freeze-drying (lyophilization) and spray-drying. A suitable lyophilization method is presented in Table A. Additional exemplary lyophilization methods can be found in U.S. Pat. Nos. 7,811,558, 8,486,617, and 8,097,403. An exemplary spray-drying method includes: combining nitrogen, as a drying gas, with a loading buffer according to the present disclosure, then introducing the mixture into GEA Mobile Minor spray dryer from GEA Processing Engineering, Inc. (Columbia Md., USA), which has a Two-Fluid Nozzle configuration, spray drying the mixture at an inlet temperature in the range of 150° C. to 190° C., an outlet temperature in the range of 65° C. to 100° C., an atomic rate in the range of 0.5 to 2.0 bars, an atomic rate in the range of 5 to 13 kg/hr, a nitrogen use in the range of 60 to 100 kg/hr, and a run time of 10 to 35 minutes. The final step in spray drying is preferentially collecting the dried mixture. The dried composition in some embodiments is stable for at least six months at temperatures that range from −20° C. or lower to 90° C. or higher.

TABLE A
Exemplary Lyophilization Protocol

| | Step | Temp. Set | | Type | Durati | | Pressure Set |
|---|---|---|---|---|---|---|---|
| Freezing Step | F1 | −50° | C. | Ramp | Var | | N/A |
| | F2 | −50° | C. | Hold | 3 | Hrs | N/A |
| Vacuum Pulldown | F3 | −50° | | Hold | Var | | N/A |
| Primary Dry | P1 | −40° | | Hold | 1.5 | Hrs | 0 mT |
| | P2 | −35° | | Ramp | 2 | Hrs | 0 mT |
| | P3 | −25° | | Ramp | 2 | Hrs | 0 mT |
| | P4 | −17° | C. | Ramp | 2 | Hrs | 0 mT |
| | P5 | 0° | C. | Ramp | 1.5 | Hrs | 0 mT |
| | P6 | 27° | C. | Ramp | 1.5 | Hrs | 0 mT |
| | P7 | 27° | C. | Hold | 16 | Hrs | 0 mT |
| Secondary Dry | S1 | 27° | C. | Hold | >8 | Hrs | 0 mT |

In some embodiments, the step of drying the mRNA agent-loaded platelets that are obtained as disclosed herein, such as the step of freeze-drying the mRNA agent-loaded platelets that are obtained as disclosed herein, includes incubating the platelets with a lyophilizing agent. In some embodiments, the lyophilizing agent is polysucrose. In some embodiments, the lyophilizing agent is a non-reducing disaccharide. Accordingly, in some embodiments, the methods for preparing mRNA agent-loaded platelets further include incubating the mRNA agent-loaded platelets with a lyophilizing agent. In some embodiments, the lyophilizing agent is a saccharide. In some embodiments, the saccharide is a disaccharide, such as a non-reducing disaccharide.

In some embodiments, the platelets are incubated with a lyophilizing agent for a sufficient amount of time and at a suitable temperature to load the platelets with the lyophilizing agent. Non-limiting examples of suitable lyophilizing agents are saccharides, such as monosaccharides and disaccharides, including sucrose, maltose, trehalose, glucose (e.g., dextrose), mannose, and xylose. In some embodiments, non-limiting examples of lyophilizing agent include serum albumin, dextran, polyvinyl pyrrolidone (PVP), starch, and hydroxyethyl starch (HES). In some embodiments, exemplary lyophilizing agents can include a high molecular weight polymer, into the loading composition. By “high molecular weight” it is meant a polymer having an average molecular weight of about or above 70 kDa. Non-limiting examples are polymers of sucrose and epichlorohydrin. In some embodiments, the lyophilizing agent is polysucrose. Although any amount of high molecular weight polymer can be used as a lyophilizing agent, it is preferred that an amount be used that achieves a final concentration of about 3% to 10% (w/v), such as 3% to 7%, for example 6%.

In some embodiments, the process for preparing a composition includes adding an organic solvent, such as ethanol, to the loading solution. In such a loading solution, the solvent can range from 0.1% to 5.0% (v/v).

Within the process provided herein for making the compositions provided herein, addition of the lyophilizing agent can be the last step prior to drying. However, in some embodiments, the lyophilizing agent is added at the same time or before the mRNA agent, the cryoprotectant, or other components of the loading composition. In some embodiments, the lyophilizing agent is added to the loading solution, thoroughly mixed to form a drying solution, dispensed into a drying vessel (e.g., a glass or plastic serum vial, a lyophilization bag), and subjected to conditions that allow for drying of the solution to form a dried composition.

An exemplary saccharide for use in the compositions disclosed herein is trehalose. Regardless of the identity of the saccharide, it can be present in the composition in any suitable amount. For example, it can be present in an amount of 1 mM to 1 M. In embodiments, it is present in an amount of from 10 mM 10 to 500 mM. In some embodiments, it is present in an amount of from 20 mM to 200 mM. In some embodiments, it is present in an amount from 40 mM to 100 mM. In various embodiments, the saccharide is present in different specific concentrations within the ranges recited above, and one of skill in the art can immediately understand the various concentrations without the need to specifically recite each herein. Where more than one saccharide is present in the composition, each saccharide can be present in an amount according to the ranges and particular concentrations recited above.

The step of incubating the platelets to load them with a cryoprotectant or as a lyophilizing agent includes incubating the platelets for a time suitable for loading, as long as the time, taken in conjunction with the temperature, is sufficient for the cryoprotectant or lyophilizing agent to come into contact with the platelets and, preferably, be incorporated, at least to some extent, into the platelets. In embodiments, incubation is carried out for about 1 minute to about 180 minutes or longer.

The step of incubating the platelets to load them with a cryoprotectant or lyophilizing agent includes incubating the platelets and the cryoprotectant at a temperature that, when selected in conjunction with the amount of time allotted for loading, is suitable for loading. In general, the composition is incubated at a temperature above freezing for at least a sufficient time for the cryoprotectant or lyophilizing agent to come into contact with the platelets. In embodiments, incubation is conducted at 37° C. In certain embodiments, incubation is performed at 20° C. to 42° C. For example, in embodiments, incubation is performed at 35° C. to 40° C. (e.g., 37° C.) for 110 to 130 (e.g., 120) minutes.

In various embodiments, the bag is a gas-permeable bag configured to allow gases to pass through at least a portion or all portions of the bag during the processing. The gas-permeable bag can allow for the exchange of gas within the interior of the bag with atmospheric gas present in the surrounding environment. The gas-permeable bag can be permeable to gases, such as oxygen, nitrogen, water, air, hydrogen, and carbon dioxide, allowing gas exchange to occur in the compositions provided herein. In some embodiments, the gas-permeable bag allows for the removal of some of the carbon dioxide present within an interior of the bag by allowing the carbon dioxide to permeate through its wall. In some embodiments, the release of carbon dioxide from the bag can be advantageous to maintaining a desired pH level of the composition contained within the bag.

In some embodiments, the container of the process herein is a gas-permeable container that is closed or sealed. In some embodiments, the container is a container that is closed or sealed and a portion of which is gas-permeable. In some embodiments, the surface area of a gas-permeable portion of a closed or sealed container (e.g., bag) relative to the volume of the product being contained in the container (hereinafter referred to as the “SA/V ratio”) can be adjusted to improve pH maintenance of the compositions provided herein. For example, in some embodiments, the SA/V ratio of the container can be at least about 2.0 cm²/mL (e.g., at least about 2.1 cm²/mL, at least about 2.2 cm²/mL, at least about 2.3 cm²/mL, at least about 2.4 cm²/mL, at least about 2.5 cm²/mL, at least about 2.6 cm²/mL, at least about 2.7 cm²/mL, at least about 2.8 cm²/mL, at least about 2.9 cm²/mL, at least about 3.0 cm²/mL, at least about 3.1 cm²/mL, at least about 3.2 cm²/mL, at least about 3.3 cm²/mL, at least about 3.4 cm²/mL, at least about 3.5 cm²/mL, at least about 3.6 cm²/mL, at least about 3.7 cm²/mL, at least about 3.8 cm²/mL, at least about 3.9 cm²/mL, at least about 4.0 cm²/mL, at least about 4.1 cm²/mL, at least about 4.2 cm²/mL, at least about 4.3 cm²/mL, at least about 4.4 cm²/mL, at least about 4.5 cm²/mL, at least about 4.6 cm²/mL, at least about 4.7 cm²/mL, at least about 4.8 cm²/mL, at least about 4.9 cm²/mL, or at least about 5.0 cm²/mL. In some embodiments, the SA/V ratio of the container can be at most about 10.0 cm²/mL (e.g., at most about 9.9 cm²/mL, at most about 9.8 cm²/mL, at most about 9.7 cm²/mL, at most about 9.6 cm²/mL, at most about 9.5 cm²/mL, at most about 9.4 cm²/mL, at most about 9.3 cm²/mL, at most about 9.2 cm²/mL, at most about 9.1 cm²/mL, at most about 9.0 cm²/mL, at most about 8.9 cm²/mL, at most about 8.8 cm²/mL, at most about 8.7 cm²/mL, at most about 8.6 cm²/mL at most about 8.5 cm²/mL, at most about 8.4 cm²/mL, at most about 8.3 cm²/mL, at most about 8.2 cm²/mL, at most about 8.1 cm²/mL, at most about 8.0 cm²/mL, at most about 7.9 cm²/mL, at most about 7.8 cm²/mL, at most about 7.7 cm²/mL, at most about 7.6 cm²/mL, at most about 7.5 cm²/mL, at most about 7.4 cm²/mL, at most about 7.3 cm²/mL, at most about 7.2 cm²/mL, at most about 7.1 cm²/mL, at most about 6.9 cm²/mL, at most about 6.8 cm²/mL, at most about 6.7 cm²/mL, at most about 6.6 cm²/mL, at most about 6.5 cm²/mL, at most about 6.4 cm²/mL, at most about 6.3 cm²/mL, at most about 6.2 cm²/mL, at most about 6.1 cm²/mL, at most about 6.0 cm²/mL, at most about 5.9 cm²/mL, at most about 5.8 cm²/mL, at most about 5.7 cm²/mL, at most about 5.6 cm²/mL, at most about 5.5 cm²/mL, at most about 5.4 cm²/mL, at most about 5.3 cm²/mL, at most about 5.2 cm²/mL, at most about 5.1 cm²/mL, at most about 5.0 cm²/mL, at most about 4.9 cm²/mL, at most about 4.8 cm²/mL, at most about 4.7 cm²/mL, at most about 4.6 cm²/mL, at most about 4.5 cm²/mL, at most about 4.4 cm²/mL, at most about 4.3 cm²/mL, at most about 4.2 cm²/mL, at most about 4.1 cm²/mL, or at most about 4.0 cm²/mL. In some embodiments, the SA/V ratio of the container can range from about 2.0 to about 10.0 cm²/mL (e.g., from about 2.1 cm²/mL to about 9.9 cm²/mL, from about 2.2 cm²/mL to about 9.8 cm²/mL, from about 2.3 cm²/mL to about 9.7 cm²/mL, from about 2.4 cm²/mL to about 9.6 cm²/mL, from about 2.5 cm²/mL to about 9.5 cm²/mL, from about 2.6 cm²/mL to about 9.4 cm²/mL, from about 2.7 cm²/mL to about 9.3 cm²/mL, from about 2.8 cm²/mL to about 9.2 cm²/mL, from about 2.9 cm²/mL to about 9.1 cm²/mL, from about 3.0 cm²/mL to about 9.0 cm²/mL, from about 3.1 cm²/mL to about 8.9 cm²/mL, from about 3.2 cm²/mL to about 8.8 cm²/mL, from about 3.3 cm²/mL to about 8.7 cm²/mL, from about 3.4 cm²/mL to about 8.6 cm²/mL, from about 3.5 cm²/mL to about 8.5 cm²/mL, from about 3.6 cm²/mL to about 8.4 cm²/mL, from about 3.7 cm²/mL to about 8.3 cm²/mL, from about 3.8 cm²/mL to about 8.2 cm²/mL, from about 3.9 cm²/mL to about 8.1 cm²/mL, from about 4.0 cm²/mL to about 8.0 cm²/mL, from about 4.1 cm²/mL to about 7.9 cm²/mL, from about 4.2 cm²/mL to about 7.8 cm²/mL, from about 4.3 cm²/mL to about 7.7 cm²/mL, from about 4.4 cm²/mL to about 7.6 cm²/mL, from about 4.5 cm²/mL to about 7.5 cm²/mL, from about 4.6 cm²/mL to about 7.4 cm²/mL, from about 4.7 cm²/mL to about 7.3 cm²/mL, from about 4.8 cm²/mL to about 7.2 cm²/mL, from about 4.9 cm²/mL to about 7.1 cm²/mL, from about 5.0 cm²/mL to about 6.9 cm²/mL, from about 5.1 cm²/mL to about 6.8 cm²/mL, from about 5.2 cm²/mL to about 6.7 cm²/mL, from about 5.3 cm²/mL to about 6.6 cm²/mL, from about 5.4 cm²/mL to about 6.5 cm²/mL, from about 5.5 cm²/mL to about 6.4 cm²/mL, from about 5.6 cm²/mL to about 6.3 cm²/mL, from about 5.7 cm²/mL to about 6.2 cm²/mL, or from about 5.8 cm²/mL to about 6.1 cm²/mL.

Gas-permeable closed containers (e.g., bags) or portions thereof can be made of one or more various gas-permeable materials. In some embodiments, the gas-permeable bag can be made of one or more polymers including fluoropolymers (such as polytetrafluoroethylene (PTFE) and perfluoroalkoxy (PFA) polymers), polyolefins (such as low-density polyethylene (LDPE), high-density polyethylene (HDPE)), fluorinated ethylene propylene (FEP), polystyrene, polyvinylchloride (PVC), silicone, and any combinations thereof.

In some embodiments, the lyophilizing agent as disclosed herein may be a high molecular weight polymer. By “high molecular weight” it is meant a polymer having an average molecular weight of about or above 70 kDa and up to 1,000,000 kDa Non-limiting examples are polymers of sucrose and epichlorohydrin (polysucrose). Although any amount of high molecular weight polymer can be used, it is preferred that an amount be used that achieves a final concentration of about 3% to 10% (w/v), such as 3% to 7%, for example 6%. Other non-limiting examples of lyoprotectants are serum albumin, dextran, polyvinyl pyrrolidone (PVP), starch, and hydroxyethyl starch (HES).

In some embodiments, the loading buffer includes an organic solvent, such as an alcohol (e.g., ethanol). In such a loading buffer, the amount of solvent can range from 0.1% to 5.0% (v/v).

In some embodiments, the mRNA agent-loaded platelets prepared as disclosed herein have a storage stability that is at least about equal to that of the platelets prior to the loading of the mRNA agent.

The loading buffer may be any buffer that is non-toxic to the platelets and provides adequate buffering capacity to the solution at the temperatures at which the solution will be exposed during the process provided herein. Thus, the buffer may include any of the known biologically compatible buffers available commercially, such as phosphate buffers, such as phosphate buffered saline (PBS), bicarbonate/carbonic acid, such as sodium-bicarbonate buffer, N-2-hydroxyethylpiperazine-N′-2-ethanesulfonic acid (HEPES), and tris-based buffers, such as tris-buffered saline (TB S). Likewise, it may include one or more of the following buffers: propane-1,2,3-tricarboxylic (tricarballylic); benzenepentacarboxylic; maleic; 2,2-dimethyl succinic; 3,3-dimethylglutaric; bis(2-hydroxyethyl)imino-tris(hydroxymethyl)-methane (BIS-TRIS); benzenehexacarboxylic (mellitic); N-(2-acetamido)imino-diacetic acid (ADA); butane-1,2,3,4-tetracarboxylic; pyrophosphoric; 1,1-cyclopentanediacetic (3,3 tetramethylene-glutaric acid); piperazine-1,4-bis-(2-ethanesulfonic acid) (PIPES); N-(2-acetamido)-2-amnoethanesulfonic acid (ACES); 1,1-cyclohexanediacetic; 3,6-endomethylene-1,2,3,6-tetrahydrophthalic acid (EMTA; ENDCA); imidazole; 2-(aminoethyl)trimethylammonium chloride (CHOLAMINE); N,N-bis(2-hydroxyethyl)-2-aminoethanesulfonic acid (BES); 2-methylpropane-1,2,3-triscarboxylic (beta-methyltricarballylic); 2-(N-morpholino)propane-sulfonic acid (MOPS); phosphoric; and N-tris(hydroxymethyl)methyl-2-amminoethane sulfonic acid (TES).

Flow cytometry can be used to obtain a relative quantification of loading efficiency by measuring the mean fluorescence intensity of the mRNA agent in the mRNA agent-loaded platelets. Platelets can be evaluated for functionality by adenosine diphosphate (ADP), collagen, arachidonic acid, thrombin receptor activating peptide (TRAP), and/or any other platelet agonist known in the art for stimulation post-loading.

In some embodiments, the mRNA agent-loaded platelets are lyophilized. In some embodiments, the mRNA agent-loaded platelets are cryopreserved.

In some embodiments, the mRNA agent-loaded platelets retain the loaded mRNA agent upon rehydration and release the mRNA agent upon stimulation by endogenous platelet activators.

In some embodiments, the mRNA loaded agents loaded into platelets are translated into their respective proteins. In some embodiments, lyophilized platelets containing mRNA loaded agents are rehydrated and the mRNA loaded agents are translated into their respective protein. In some embodiments, cryopreserved platelets containing mRNA loaded agents are rehydrated and the mRNA loaded agents (e.g., mRNA) are translated into their respective protein.

In some embodiments, the dried platelets (such as freeze-dried platelets) retain the loaded mRNA agent upon rehydration and release the mRNA agent (e.g., mRNA) upon stimulation by endogenous platelet activators. In some embodiments, the dried platelets (such as freeze-dried platelets) retain the loaded mRNA agent and/or its respective protein. In some embodiments, at least about 10%, such as at least about 20%, such as at least about 30% of the mRNA agent is retained. In some embodiments, from about 10% to about 20%, such as from about 20% to about 30% of the mRNA agent is retained.

In some embodiments, the dried platelets (such as freeze-dried platelets) retain the loaded mRNA agent and or its respective protein and upon rehydration the mRNA agents are translated and their respective proteins are released upon stimulation by endogenous platelet activators. In some embodiments, at least about 10%, such as at least about 20%, such as at least about 30% of the mRNA agent is retained. In some embodiments, from about 10% to about 20%, such as from about 20% to about 30% of the mRNA agent is retained.

Any suitable mRNA agent (e.g., mRNA) may be loaded in a platelet. An example of an mRNA agent that may be loaded in a platelet includes, but is not limited, to CleanCAP Cyanine 5 EGFP mRNA (TriLink Cat. #L-7701; 1 mg/ml).

Various agents and/or procedures may be used to load the platelets with a mRNA agent. In some embodiments, the platelets are loaded with a mRNA agent previously incubated with a cationic lipid such as, without limitation, lipofectamine.

Exemplary protocols that employ the foregoing agents or procedures are shown below:

### Lipofectamine Transfection

### Background

Lipofectamine is a cationic lipid; the Lipofectamine positively charged head group interacts with the negatively charged phosphate backbone of nucleic acids to facilitate transfection. Cellular internalization of the nucleic acid is achieved by incubating cells with the complexed Lipofectamine and nucleic acid.

### Protocol

As described here and in the Examples below, prepare the Lipofectamine and mRNA agent in aqueous buffer at room temperature. Incubate the complexed Lipofectamine and mRNA agent with platelets for at least 30 minutes. Transfected platelets may be lyophilized to create Thrombosomes with an mRNA agent (e.g., mRNA). Fluorescently labeled mRNA agent can be detected via flow cytometry and visualized using fluorescence microscopy. This method of loading is applicable to mRNA.

In some embodiments, mRNA agent-loaded platelets, mRNA agent-loaded platelet derivatives, or mRNA agent-loaded thrombosomes may shield the mRNA agent from exposure in circulation, thereby reducing or eliminating systemic toxicity (e.g. cardiotoxicity) associated with the mRNA agent. In some embodiments, mRNA agent-loaded platelets, mRNA agent-loaded platelet derivatives, or mRNA agent-loaded thrombosomes may also protect the mRNA agent from metabolic degradation or inactivation. In some embodiments, mRNA agent delivery with mRNA agent-loaded platelets, mRNA agent-loaded platelet derivatives, or mRNA agent-loaded thrombosomes may therefore be advantageous in treatment of diseases such as cancer, since mRNA agent-loaded platelets, mRNA agent-loaded platelet derivatives, or mRNA agent-loaded thrombosomes facilitate targeting of cancer cells while mitigating systemic side effects. In some embodiments, mRNA agent-loaded platelets, mRNA agent-loaded platelet derivatives, or mRNA agent-loaded thrombosomes may be used in any therapeutic setting in which expedited healing process is required or advantageous.

In some embodiments, provided herein is a method of treating a disease as disclosed herein, comprising administering mRNA agent-loaded platelets, mRNA agent-loaded platelet derivatives, or mRNA agent-loaded thrombosomes as disclosed herein. In some embodiments, provided herein is a method of treating a disease as disclosed herein, comprising administering cold stored, room temperature stored, cryopreserved thawed, rehydrated, and/or lyophilized platelets, platelet derivatives, or thrombosomes as disclosed herein. In some embodiments, the disease is cancer. In some embodiments, the disease is Traumatic Brain injury. In some embodiments, the disease is idiopathic thrombocytomenic purpura (ITP). In some embodiments, the disease is thrombotic thrombocytopenic purpura (TTP). In some embodiments, the disease is an inherited disorder. In some embodiments, the disease is heart disease. In some embodiments, the disease is kidney disease. In some embodiments, the disease is a nervous system development disease. In some embodiments, the disease is hemostasis. In some embodiments, the disease is obesity.

Examples of diseases (therapeutic indications) that may be treated with the RNA agent-loaded platelets are as follows:

Therapeutic indications

Acute lymphoblastic leukemia (ALL)
Acute myeloid leukemia (AML)
Breast cancer (can also be used as an adjuvant
therapy for metastasized breast cancer post-surgery)
Gastric cancer
Hodgkin lymphoma
Neuroblastoma
Non - Hodgkin lymphoma
Ovarian cancer
Cervical cancer
Small cell lung cancer
Non-small cell lung cancer (NSCLC)
Soft tissue and bone sarcomas
Thyroid cancer
Transitional cell bladder cancer
Wilms tumor
Neuroendocrine tumors
Pancreatic cancer
Multiple myeloma
Renal cancer
Glioblastoma
Prostate cancer
Sarcoma
Colon cancer
Melanoma
Colitis
Chronic inflammatory demyelinating polyneuropathy
Guillain - Barre syndrome
Immune Thrombocytopenia
Kawasaki disease
Lupus
Multiple Sclerosis
Myasthenia gravis
Myositis
Cirrhosis with refractory ascites
Hepatorenal syndrome (used in combination with
vasoconstrictive drugs)
Nephrotic syndrome (for patient with albumin <2
g/dL with hypovolaemia and/or pulmonary edema)
Organ transplantation
Paracentesis
Hypovolemia
Aneurysms
Artherosclerosis
Cancer
Cardiovascular diseases (post - myocardial infarction
remodeling, cardiac regeneration, cardiac fibrosis,
viral myocarditis, cardiac hypertrophy, pathological
cardiac remodeling)
Genetic disorders
Infectious diseases
Metabolic diseases
Neoangiogenesis
Opthalmic conditions (retinal angiogenesis, ocular
hypertension, glaucoma, diabetic macular edema,
diabetic retinopathy, macular degeneration)
Hypercholesterolemia
Pulmonary hypertension

Examples of mRNA agent and therapeutic indications for mRNA agent(s) to be loaded into platelets (e.g., translated mRNA agents into their respective proteins) are as follows:

| mRNA Agent | Therapeutic indications |
|---|---|
| mRNA | Aneurysms |
| | Artherosclerosis |
| | Cancer |
| | Cardiovascular diseases (post - myocardial infarction |
| | remodeling, cardiac regeneration, cardiac fibrosis, |
| | viral myocarditis, cardiac hypertrophy, pathological |
| | cardiac remodeling) |
| | Genetic disorders |
| | Metabolic diseases |
| | Neoangiogenesis |
| | Opthalmic conditions (retinal angiogenesis) |
| | Pulmonary hypertension |

In some embodiments, incubation is performed at 22° C. using a platelet to cationic lipid-mRNA agent volume ratio of about 10:1, using different incubation periods.

While the embodiments of the invention are amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### EXAMPLES

### Example 1. mRNA-Loaded Platelets

Fresh platelets were tested for their ability to uptake mRNA via lipofectamine transfection with Lipofectamine™ MessengerMAX™ (ThermoFisher Cat. # LMRNA003) with varying concentrations of suspended platelets and lipofectamine. A total of nine mRNA loaded platelet samples were prepared including 3 different platelet suspension concentrations (4 k/μl, 40 k/μl, and 100 k/μl) with 3 different concentrations of Lipofectamine™ MessengerMAX™ (0.0 μl, 0.75 μl, and 1.5 μl, each in 25 μl HBS) as described in Protocol 1. Each of the nine samples also contained 0.25 μg of CleanCAP Cyanine 5 EGFP mRNA (TriLink Cat. #L-7701; 1 mg/ml) also described in Protocol 1. All nine samples were assayed by flow cytometry for single platelet size (FSC/SSC), CY5 mean fluorescence intensity, and CY5 positive platelets, and absolute count at 30 minutes (Table 2) and at 120 minutes (Table 3).

TABLE 1
Loading conditions

| All 0.25 μg | 4k/μL, | 40k/μL, | 100k/μL |
|---|---|---|---|
| mRNA, 0.5 mL | (Platelets) | (Platelets) | (Platelets) |
| volume | 20x dilution | 200x dilution | 500x dilution |
| 0.75 μl | A | B | C |
| Lipofectamine | | | |
| 1.50 μl | D | E | F |
| Lipofectamine | | | |
| No Transfection | G | H | I |
| (HBS only) | | | |

TABLE 2
30 Minutes Transfection

| | Platelets | Platelet Size | Platelet Cy5 | Cy5 Positive |
|---|---|---|---|---|
| Sample | per μL | (FSC-H) | MFI | Platelets |
| A | 3,520 | 248,128 | 7,781 | 68.70% |
| B | 33,893 | 283,856 | 3,561 | 35.27% |
| C | 91,741 | 287,321 | 1,444 | 20.20% |
| D | 3,855 | 284,302 | 5,131 | 99.32% |
| E | 32,424 | 282,348 | 2,620 | 62.96% |
| F | 85,964 | 283,569 | 1,063 | 33.94% |
| G | 5,615 | 267,719 | 279 | 0.33% |
| H | 38,377 | 274,645 | 313 | 0.92% |
| I | 89,701 | 276,510 | 318 | 0.99% |

TABLE 3
120 Minutes Transfection

| | Platelets | Platelet Size | Platelet Cy5 | Cy5 Positive |
|---|---|---|---|---|
| Sample | per μL | (FSC-H) | MFI | Platelets |
| A | 2,993 | 222,212 | 14,365 | 84.27% |
| B | 33,563 | 268,001 | 3,910 | 40.60% |
| C | 87,586 | 274,128 | 1,525 | 25.48% |
| D | 2,893 | 210,797 | 13,604 | 99.77% |
| E | 29,564 | 280,217 | 3,097 | 69.43% |
| F | 83,637 | 273,206 | 1,085 | 38.40% |
| G | 4,243 | 256,313 | 278 | 0.34% |
| H | 39,540 | 272,678 | 316 | 0.93% |
| I | 92,597 | 274,686 | 313 | 0.89% |

TABLE 4
HBS:

| | Concentration |
|---|---|
| Component | (mg/mL) |
| NaCl | 8.77 |
| HEPES | 2.38 |
| pH | 6.6-6.8 |

TABLE 5
Loading Buffer:

| | Concentration |
|---|---|
| | (mg/mL, except where |
| Component | otherwise indicated) |
| NaCl | 4.38 |
| KCl | 0.36 |
| HEPES | 2.26 |
| NaHCO₃ | 1.01 |
| Dextrose | 0.54 |
| Trehalose | 34.23 |
| Ethanol | 1.00% (v/v) |
| pH | 6.6-6.8 |

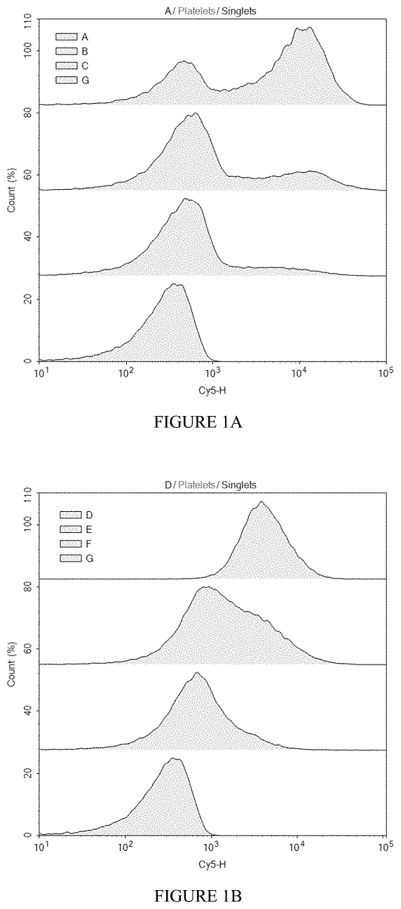

FIGS. 1A and 1B are flow cytometry histograms showing detection of Cy5-H after 30 minutes of incubation for samples A through I shown in Table 1 indicating that platelets can be transfected with mRNA (CleanCAP Cyanine 5 EGFP mRNA) and lipofectamine transfection reagents, Lipofectamine™ MessengerMAX™.

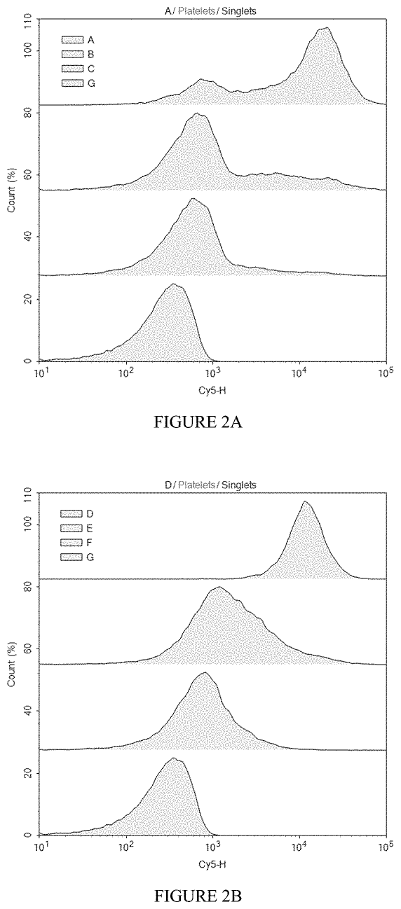

Cy5-H was detected after 120 minutes of incubation for samples A through I shown in Table 1 indicating that platelets can be transfected with mRNA with lipofectamine transfection reagents, Lipofectamine™ MessengerMAX™ (FIGS. 2A and 2B).

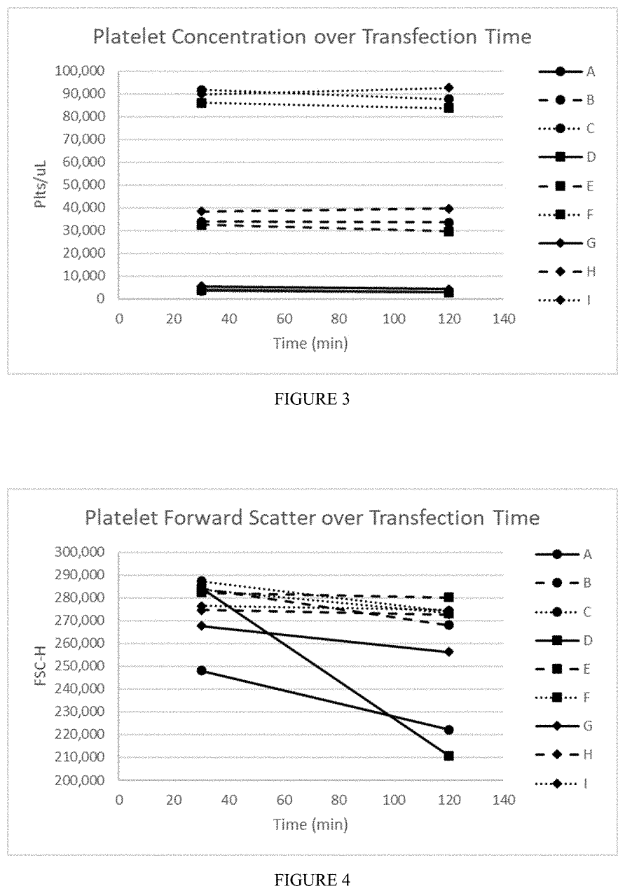

mRNA loaded platelet concentration (Plts/μl) was measured over 120 minutes of incubation time at 30 minutes and 120 minutes (FIG. 3). The three platelet suspension dilutions (4 k/μl, 40 k/μl, and 100 k/μl) transfected with varying concentrations of Lipofectamine™ MessengerMAX™ show no effect on platelet count after 120 minutes of incubation.

Platelet forward scatter (FSC-H) was measured over 120 minutes of an incubation time at 30 minutes and 120 minutes (FIG. 4). For most samples tested FSC-H decreases between the 30 minute measurement and the 120 minute measurement. For samples with low platelet concentration (4 k/μl) and a high lipofectamine concentration (0.75 μl, 1.5 μl), FSC-H decreased significantly (Samples A and D in Table 1).

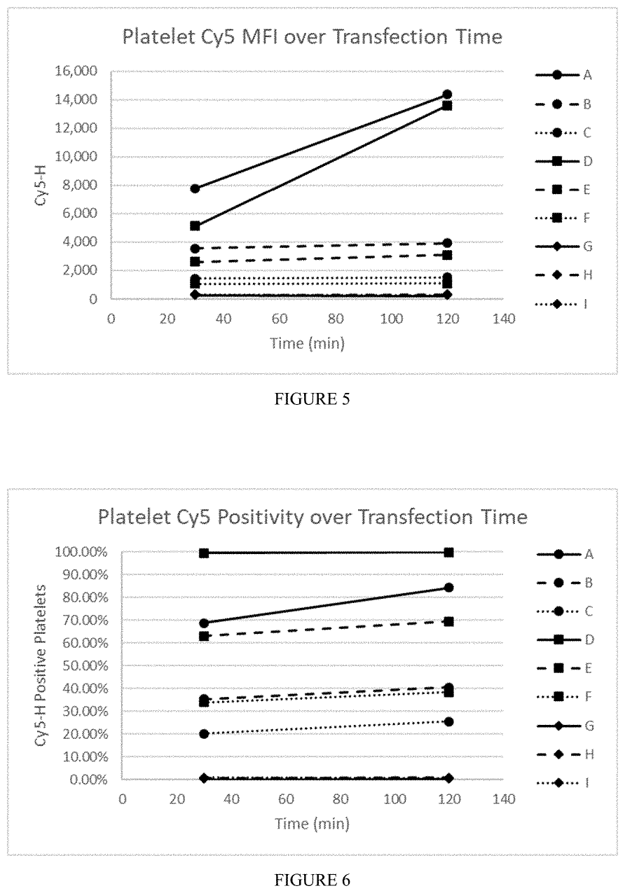

Cy5 mean fluorescent intensity (MFI) was measured over 120 minutes of an incubation time at 30 minutes and 120 minutes (FIG. 5). For samples tested with high platelet concentrations, 40 k/μl and 100 k/μl, Cy5 MFI was stable through two hours of incubation time. For tested samples with the lowest platelet concentration, 4 k/μl, Cy5 MFI increased from 30 minutes to 120 minutes incubation. All flow cytometry was performed with the Acea NovoCyte® (configuration 3005) flow cytometer for all flow data described herein.

Platelet Cy5-H positivity was measured over 120 minutes of an incubation time at 30 minutes and 120 minutes (FIG. 6). For all samples tested Cy5 positivity is stable beyond 30 minutes and showed slight increases, except sample A, in platelets over transfection time.

Protocol 1: Loading Platelets with mRNA

The starting apheresis platelet material were washed in loading buffer via centrifugation to generate a master platelet stock at 1,000 platelets/μl (minimum 1 mL) in loading buffer. The washed platelets were diluted into three pools, 4 k/μL (samples A, D, and G), 40 k/μl (samples B, E, and H), and 100 k/μl (samples C, F, and I) (Table 1). Each sample contained 500 μl.

The lipofectamine transfection reagent, Lipofectamine™ MessengerMAX™ (ThermoFisher Cat. # LMRNA003) was prepared in 6 tubes, each tube containing 25 μl HBS. The six tubes were divided into two groups: 3 tubes containing 0.75 μl Lipofectamine™ MessengerMAX™ and 3 tubes containing 1.50 μl Lipofectamine™ MessengerMAX™. All tubes were incubated for 10 minutes at room temperature.

150 μL of HBS including 1.5 μl (1.5 μg) of Cy5-linked EGFP mRNA (TriLink Cat. # L-7701; 1 mg/mL) was prepared and incubated for 10 minutes at room temperature.

25 μl of the 150 μL of HBS including 1.5 μl (1.5 μg) of Cy5-linked EGFP mRNA mixture were allocated to each of the 3 tubes containing 0.75 μl Lipofectamine™ MessengerMAX™ and each of the 3 tubes containing 1.50 μl Lipofectamine™ MessengerMAX™ for a total volume of 50 μl and a concentration of Cy5-linked EGFP mRNA of 0.25 μg. Each tube was mixed and incubated at room temperature for 5 minutes.

Each of the six tubes containing 25 μl of HBS including 1.5 μl (1.5 μg) of Cy5-linked EGFP mRNA and including one of the varying concentrations of 25 μl of Lipofectamine™ MessengerMAX™ (either at a concentration of 0.75 μl or 1.50 μl) were added to the designated platelet suspension for a final volume of 550 μl. Negative control, “no transfection,” samples were prepared by adding an equivalent volume, 50 μl, of HBS to each of the designated platelet suspensions 4 k/μL, 40 k/μl, and 100 k/μl.

The platelet suspensions and transfection reagents were incubated for at least 2 hours at 37° C. with protection from ambient light. Incubation was conducted in a 2.0 mL snap-top microcentrifuge tube.

Platelet samples were analyzed at two time points, 30 minutes and 120 minutes, by flow cytometry (e.g., NovoCyte Flow Cytometer) for FSC/SSC (forward scatter/side scatter), Cy5 label detection, and absolute count.

Designated platelet suspension samples at 4 k/μl were diluted 2×. Designated platelet suspension samples at 40 k/μl were diluted 20×. Designated platelet suspension samples at 100 k/μl were diluted 50×.

Platelet suspension sample were assessed by eye for aggregation events.

### Example 2: mRNA Loaded Platelets

Fresh platelets were tested for their ability to uptake mRNA with varying concentrations of lipofectamine Lipofectamine™ MessengerMAX™ (ThermoFisher Cat. # LMRNA003) and varying concentrations of CleanCAP Cyanine 5 EGFP mRNA (TriLink L-7701; 1 mg/mL). All samples tested contained a suspended platelet dilution of about 40 k/μl in 500 μl in loading buffer.

Nine experimental conditions were tested with three concentrations of CleanCAP Cyanine 5 EGFP mRNA (0.25 μg, 0.50 μg, and 0.85 μg) and three concentrations of Lipofectamine™ MessengerMAX™ (1.50 μl, 3.00 μl, and 5.00 μl). The CleanCAP Cyanine 5 EGFP mRNA and Lipofectamine™ MessengerMAX mixtures were prepared according to Protocol 2. All nine samples were assayed by flow cytometry for single platelet size (FSC/SSC), CY5 mean fluorescent intensity, FITC, and absolute count at 30 minutes (Table 7) and 120 minutes (Table 8). A control sample containing no CleanCAP Cyanine 5 EGFP mRNA was included as well (Table 6. Sample J).

TABLE 6
Loading conditions

| All ~40k/μl | | | |
|---|---|---|---|
| plts, 0.5 mL | 1.50 μl | 3.00 μl | 5.00 μl |
| volume | Lipofectamine | Lipofectamine | Lipofectamine |
| 0.25 μg mRNA | A | B | C |
| 0.50 μg mRNA | D | E | F |
| 0.85 μg mRNA | G | H | I |
| No Transfection | J | N/A | N/A |
| Control | | | |
| (HBS only) | | | |

TABLE 7
30 Minutes Transfection

| | Platelets | Platelet Size | Platelet Cy5 | Cy5 Positive |
|---|---|---|---|---|
| Sample | per μL | (FSC-H) | MFI | Platelets |
| A | 35,767 | 245,993 | 1,465 | 73.45% |
| B | 27,419 | 300,997 | 3,572 | 99.00% |
| C | 21,201 | 282,855 | 3,919 | 98.45% |
| D | 32,539 | 252,273 | 2,303 | 24.96% |
| E | 31,145 | 270,048 | 3,930 | 96.48% |
| F | 18,989 | 307,676 | 5,307 | 99.48% |
| G | 29,827 | 232,654 | 1,799 | 68.71% |
| H | 26,943 | 252,389 | 4,563 | 31.85% |
| I | 23,416 | 268,113 | 5,733 | 81.64% |
| J | 28,991 | 243,790 | 256 | 0.96% |

TABLE 8
120 Minutes Transfection

| | Platelets | Platelet Size | Platelet Cy5 | Cy5 Positive |
|---|---|---|---|---|
| Sample | per μL | (FSC-H) | MFI | Platelets |
| A | 33,720 | 238,126 | 1,656 | 78.62% |
| B | 17,703 | 277,375 | 5,705 | 99.40% |
| C | 12,863 | 175,295 | 7,720 | 99.70% |
| D | 29,564 | 237,394 | 2,891 | 35.39% |
| E | 20,747 | 257,967 | 6,141 | 96.87% |
| F | 12,320 | 210,576 | 12,153 | 99.77% |
| G | 29,658 | 225,011 | 3,621 | 88.12% |
| H | 25,442 | 239,226 | 6,298 | 37.56% |
| I | 15,946 | 251,571 | 11,554 | 88.66% |
| J | 26,755 | 229,117 | 248 | 0.65% |

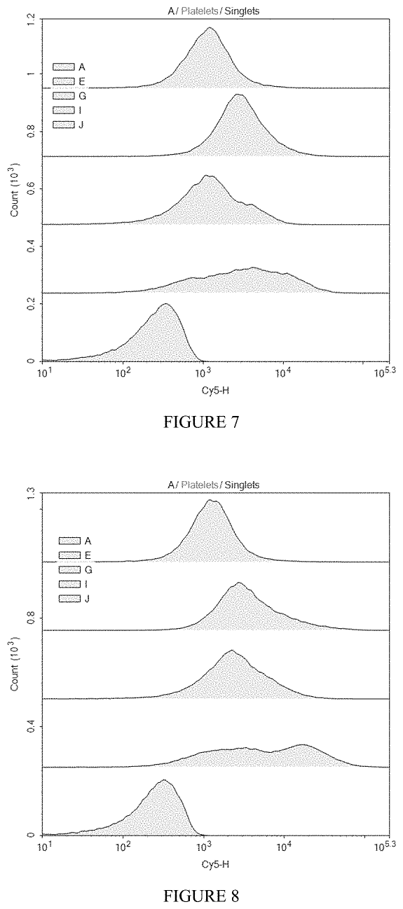

A flow cytometry histogram showing detection of Cy5-H after 30 minutes of incubation for samples A, E, G, I, and J (negative control) in Table 6 indicates that platelets can be transfected with mRNA (FIG. 7). Additionally, a flow cytometry histogram showing detection of Cy5-H after 120 minutes of incubation for samples A, E, G, I, and J (negative control) in Table 4 indicates that platelets can be transfected with mRNA (FIG. 8).

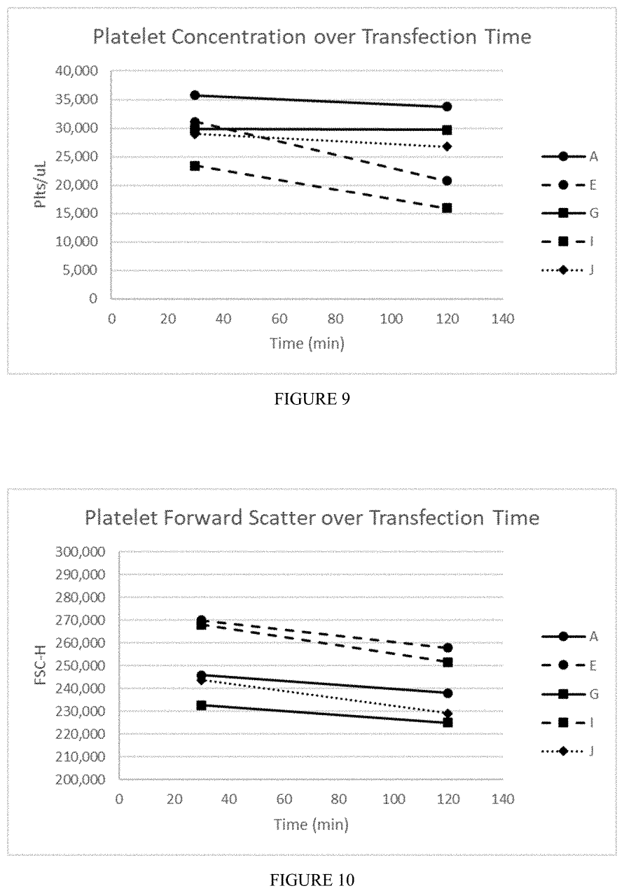

Single platelet count was measured over 120 minutes of incubation time at 30 minutes and 120 minutes for samples A, E, G, I, and J (negative control) in Table 6. Samples A, E, and J showed slight decreases in single platelet count, while samples E and I showed significant decreases in platelet count between measured time points 30 minutes and 120 minutes (FIG. 9).

Single platelet FSC-H was measured over 120 minutes of incubation time at 30 minutes and 120 minutes for samples A, E, G, I, and J (negative control) in Table 6. All tested samples showed a decrease in single platelet FSC-H between measured time points 30 minutes and 120 minutes, respectively. (FIG. 10)

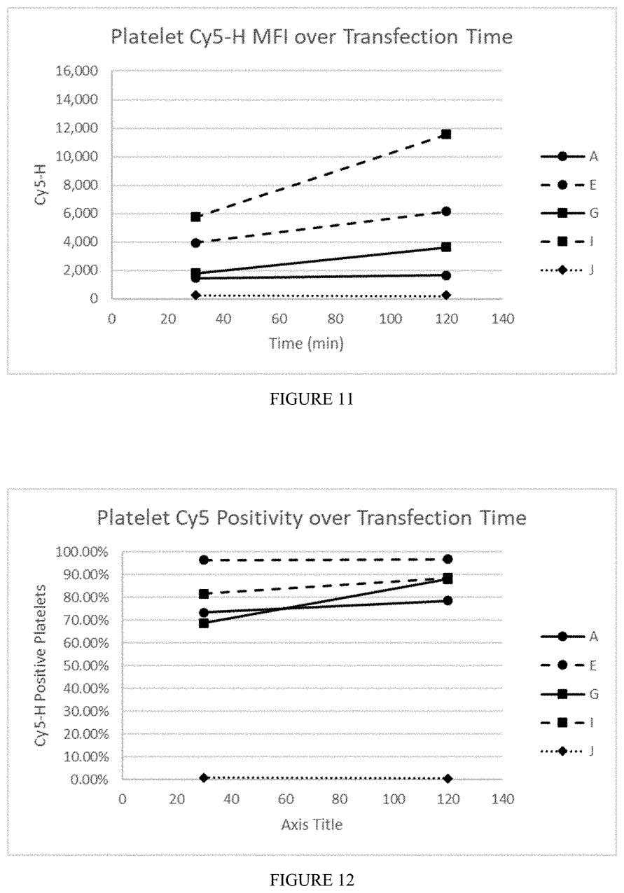

Singlet mRNA loaded platelet Cy5-H was measured over 120 minutes of incubation time at 30 minutes and 120 minutes for samples A, E, G, I, and J (negative control) in Table 6. Samples A, E, and G showed slight increases in mRNA loaded platelet Cy5-H. Sample I showed a significant increase in platelet Cy5-H measured between measured time points 30 minutes and 120 minutes, respectively (FIG. 11).

Singlet mRNA loaded platelet Cy5 positivity was measured over 120 minutes of incubation time at 30 minutes and 120 minutes for samples A, E, G, and I in Table 6. Samples A, E, and I showed slight increases in Cy5 positivity. Sample G showed a significant increase between measured time points 30 minutes and 120 minutes, respectively (FIG. 12).

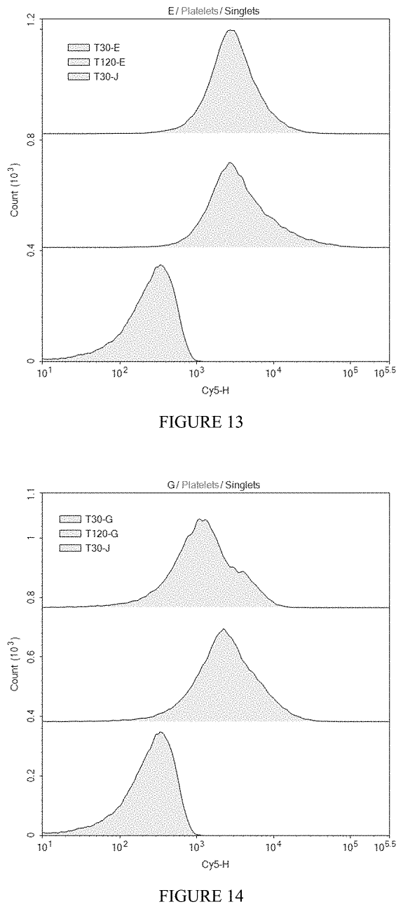

A flow cytometry histogram showing detection of Cy5-H in sample E from Table 6 after 30 minutes and 120 minutes of incubation time. Sample J (negative control) is also shown (bottom most peak) (FIG. 13).

A flow cytometry histogram showing detection of Cy5-H in sample G from Table 4 after 30 minutes and 120 minutes of incubation time. Sample J (negative control) is also shown (bottom most peak) (FIG. 14).

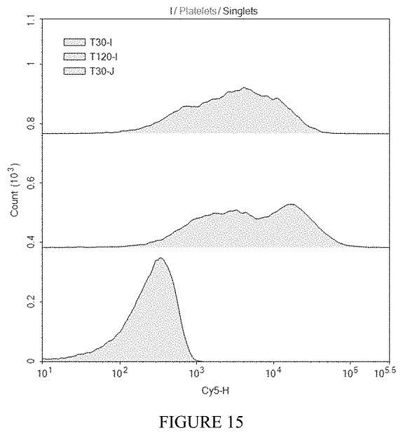

A flow cytometry histogram showing detection of Cy5-H in sample I from Table 4 after 30 minutes and 120 minutes of incubation time. Sample J (negative control) is also shown (bottom most peak) (FIG. 15)

Additionally, in accordance with Protocol 2, all test samples were checked by eye for platelet aggregation. No platelet aggregates were visible by eye at any timepoint.

Without being limited by any theory, the results from Sample G indicate that there may be an upper threshold to the quantity of mRNA that can be loaded into platelets. Additionally, Sample C and Sample F had apparent high toxicity, based on platelet count and FSC (data not shown) at 30 minutes. Further, Sample I demonstrated similar high toxicity at 120 minutes of incubation (FIGS. 9 and 10).

Test samples D and H demonstrated poor mRNA uptake with only a small proportion of the platelet population testing positive for Cy5 mRNA (Tables 7 and 8), while after 120 minutes of incubation Sample B had a dramatically reduced platelet count and the FSC had deteriorated significantly (Tables 7 and 8).

Protocol 2: Loading Platelets with mRNA

The starting apheresis platelet material were washed in loading buffer via centrifugation to generate a master platelet stock at >1,000,000 platelets/μl (minimum 1 mL) in loading buffer described herein. The washed platelets were further diluted to a concentration of 50,000 platelets/μl using AcT counts as a guide. 500 μl were aliquoted for each test condition into a 2.0 mL snap top microcentrifuge tube.

The lipofectamine transfection reagent, Lipofectamine™ MessengerMAX™ (ThermoFisher Cat. # LMRNA003) was prepared in 9 tubes, each containing 25 μl total volume HBS as described herein. The 9 tubes were divided into three groups: 3 tubes containing 1.50 μl Lipofectamine MessengerMAX™, 3 tubes containing 3.0 μl of Lipofectamine MessengerMAX™, and 3 tubes containing 5.0 μl of Lipofectamine MessengerMAX™. All tubes were incubated at room temperature for 10 minutes.

Separately, Cy5-linked EFGP mRNA ((TriLink L-7701; 1 mg/mL) was added to 3 tubes containing 75 μl of HBS. One tube received 0.75 μl (0.75 ug) of Cy5-linked EGFP mRNA, one tube received 1.50 μl (1.50 μg) of Cy5-linked EGFP mRNA, and one tube received 2.55 μl (2.55 μg) of Cy5-linked EGFP mRNA. All tubes were mixed and incubated at room temperature for 10 minutes.

25 μl of each Cy5-linked EGFP mRNA concentration in 75 μl HBS were added to each of the concentrations of tubes containing the varying concentrations of Lipofectamine MessengerMAX™. For example, three aliquots of 25 μl of the 0.75 μl (0.75 μg) containing Cy5-linked EGFP mRNA were added to one tube each of the Lipofectamine MessengerMAX™ containing 1.5 μL, 3.0 μl, and 5.0 μl in HBS, respectively. Each tube was mixed and incubated at room temperature for 5 minutes.

Each of the nine tubes containing 25 μl of HBS including one of the varying concentrations of Cy5-linked EGFP mRNA (0.75 pg. 1.5 μg, and 2.55 μg, respectively) and 25 μl in HBS including one of the varying concentrations of Lipofectamine™ MessengerMAX™ (1.50 μl, 3.0 μl, and 5.0 μl, respectively) were added to 500 μl of diluted platelets at about 40 k platelets/μl for a final volume of 550 μl. Negative control, “no transfection,” samples were prepared by adding an equivalent volume, 50 of HBS to each of the test conditions without the Lipofectamine™ MessengerMAX™ transfection reagent.

The platelet suspensions and transfection reagents were incubated for at least 2 hours at 37° C. with protection from ambient light. Incubation was conducted in a 2.0 mL snap-top microcentrifuge tube.

Platelet samples were analyzed at two time points, 30 minutes and 120 minutes, by flow cytometry (e.g., NovoCyte Flow Cytometer) for FSC/SSC (forward scatter/side scatter), Cy5 label detection, FITC, and absolute count after 20× dilution in HBS.

Platelet suspension samples were assessed by eye for aggregation events.

### EXEMPLARY EMBODIMENTS

1) A method of preparing mRNA agent-loaded platelets, comprising:
   treating platelets with a mRNA agent
   a cationic transfection reagent;
   and a loading buffer comprising a salt, a base, a loading agent, and optionally at least one organic solvent,
   to form the mRNA agent-loaded platelets.
2) A method of preparing mRNA agent-loaded platelets, comprising:
   a) providing platelets;
      and
   b) treating the platelets with a mRNA agent; a cationic transfection reagent; and a loading buffer comprising a salt, a base, a loading agent, and optionally at least one organic solvent
      to form the mRNA agent-loaded platelets.
3) The method of any one of the preceding embodiments, wherein the platelets are treated with the mRNA agent and with the loading buffer sequentially, in either order.
4) The method of any one of the preceding embodiments, wherein the platelets are treated with the mRNA agent and with the cationic transfection reagent sequentially, in either order.
5) A method of preparing mRNA agent-loaded platelets, comprising:
   (1) treating platelets with an mRNA agent to form a first composition; and
   (2) treating the first composition with a loading buffer comprising a salt, a base, a loading agent, and optionally at least one organic solvent, to form the mRNA agent-loaded platelets.
6) The method of embodiment 5, wherein the first composition is treated with a cationic transfection reagent.
7) The method of embodiment 6, wherein the first composition treated with the cationic transfection reagent is treated with a loading buffer comprising a salt, a base, a loading agent, and optionally at least one organic solvent, to form the mRNA agent-loaded platelets.
8) A method of preparing mRNA agent-loaded platelets, comprising:
   (1) treating the platelets with a loading buffer comprising a salt, a base, a loading agent, and optionally at least one organic solvent to form a first composition; and
   (2) treating the first composition with a mRNA agent, to form the mRNA agent-loaded platelets.
9) The method of embodiment 8, wherein the first composition is treated with a cationic transfection reagent.
10) The method of embodiment 9, wherein the first composition treated with the cationic transfection reagent is treated with a mRNA agent, to form the mRNA agent-loaded platelets.
11) The method of embodiment 1 or 2, wherein the platelets are treated with the mRNA agent and with the loading buffer concurrently.
12) The method of embodiment 1 or 2, wherein the platelets are treated with the mRNA agent and with the cationic transfection reagent concurrently.
13) A method of preparing mRNA agent-loaded platelets, comprising:
   treating the platelets with a mRNA agent in the presence of a cationic transfection reagent and a loading buffer comprising a salt, a base, a loading agent, and optionally at least one organic solvent to form the mRNA agent-loaded platelets.
14) The method of any one of the preceding embodiments, wherein the platelets are pooled from a plurality of donors.
15) A method of preparing mRNA agent-loaded platelets comprising:
   A) pooling platelets from a plurality of donors; and
   B) treating the platelets from step (A) with a mRNA agent; a cationic transfection reagent; and with a loading buffer comprising a salt, a base, a loading agent, and optionally at least one organic solvent, to form the mRNA agent-loaded platelets.
16) A method of preparing mRNA agent-loaded platelets comprising
   A) pooling platelets from a plurality of donors; and
   B)
      (1) treating the platelets from step (A) with a mRNA agent to form a first composition; and
      (2) treating the first composition with a loading buffer comprising a salt, a base, a loading agent, and optionally at least one organic solvent, to form the mRNA agent-loaded platelets.
17) The method of embodiment 16, wherein the first composition is treated with a cationic transfection reagent.
18) The method of embodiment 17, wherein the first composition treated with the cationic transfection agent is treated with a loading buffer comprising a salt, a base, a loading agent and optionally at least one organic solvent, to form the mRNA agent-loaded platelets.
19) A method of preparing mRNA agent-loaded platelets comprising
   A) pooling platelets from a plurality of donors; and
   B)
      (1) treating the platelets from step (A) with a loading buffer comprising a salt, a base, a loading agent, and optionally at least one organic solvent, to form a first composition; and
      (2) treating the first composition with a mRNA agent to form the mRNA agent-loaded platelets.
20) The method of embodiment 19, wherein the first composition is treated with a cationic transfection reagent.
21) The method of embodiment 20, wherein the first composition treated with the cationic transfection agent is treated with a loading buffer comprising a salt, a base, a loading agent, and optionally at least one organic solvent, to form the mRNA agent-loaded platelets.
22) A method of preparing mRNA agent-loaded platelets comprising
   A) pooling platelets from a plurality of donors; and
   B) treating the platelets with a mRNA agent in the presence of a cationic transfection reagent and a loading buffer comprising a salt, a base, a loading agent, and optionally at least one organic solvent, to form the mRNA agent-loaded platelets.
23) The method of any one of the preceding embodiments, wherein the loading buffer comprises optionally at least one organic solvent.
24) The method of any one of the preceding embodiments, wherein the loading agent is a monosaccharide or a disaccharide.
25) The method of any one of the preceding embodiments, wherein the loading agent is sucrose, maltose, dextrose, trehalose, glucose, mannose, or xylose.
26) The method of any one of the preceding embodiments, wherein the platelets are isolated prior to a treating step.
27) The method of any one of the preceding embodiments, wherein the platelets are selected from the group consisting of fresh platelets, stored platelet, and any combination thereof 28) The method of any one of the preceding embodiments, wherein the cationic transfection reagent is a cationic lipid transfection reagent.
29) The method of any one of the preceding embodiments, wherein the mRNA agent comprises mRNA.
30) The method of any one of the preceding embodiments, wherein the platelets are loaded with the mRNA agent in a period of time of 1 minute to 48 hours.
31) The method of any one of the preceding embodiments, wherein the concentration of mRNA agent in the mRNA agent-loaded platelets is from about 0.1 nM to about 10 μM.
32) The method of any one of the preceding embodiments, wherein the concentration of mRNA agent in the mRNA agent-loaded platelets is about 100 nM.
33) The method of any one of the preceding embodiments, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), or combinations thereof.
34) The method of any one of the preceding embodiments, further comprising cold storing, cryopreserving, freeze-drying, thawing, rehydrating, and combinations thereof the mRNA agent-loaded platelets.
35) The method of embodiment 34, wherein the drying step comprises freeze-drying the mRNA agent-loaded platelets.
36) The method of embodiment 34 or 35, further comprising rehydrating the mRNA agent-loaded platelets obtained from the drying step.
37) mRNA agent-loaded platelets prepared by the method of any one of the preceding embodiments.
38) Rehydrated mRNA agent-loaded platelets prepared by a method comprising rehydrating the mRNA agent-loaded platelets of embodiment 37.
39) The method of any one of the preceding embodiments, wherein the method does not comprise treating the platelets with an organic solvent.
40) The method of any one of embodiments 5 to 10 or 16 to 21, wherein the method does not comprise treating the first composition with an organic solvent.

1. A method of preparing mRNA agent-loaded platelets, comprising:
contacting platelets with a mRNA agent complexed with a cationic transfection reagent;
and a loading buffer comprising a salt, a base, a loading agent, and optionally at least one organic solvent,
to form the mRNA agent-loaded platelets.

2. (canceled)

3. The method of claim 1, wherein the platelets are contacted with the mRNA agent and with the loading buffer sequentially, in either order, or concurrently.

4. The method of claim 1, wherein the platelets are contacted with the mRNA agent and with the cationic transfection reagent sequentially, in either order, or concurrently.

5. The method of claim 1, wherein the platelets are pooled from a plurality of donors.

6. A method of preparing mRNA agent-loaded platelets comprising
A) pooling platelets from a plurality of donors; and
B) contacting the platelets from step (A) with a mRNA agent complexed with a cationic transfection reagent; and with a loading buffer comprising a salt, a base, a loading agent, and optionally at least one organic solvent, to form the mRNA agent-loaded platelets.

7. The method of claim 1, wherein the loading buffer comprises optionally at least one organic solvent.

8. The method of claim 1, wherein the loading agent is a monosaccharide or a disaccharide.

9. The method of claim 1, wherein the loading agent is sucrose, maltose, dextrose, trehalose, glucose, mannose, or xylose.

10. The method of claim 1, wherein the platelets are isolated prior to a contacting step.

11. The method of claim 1, wherein the platelets are selected from the group consisting of fresh platelets, stored platelets, and any combination thereof.

12. The method of claim 1, wherein the cationic transfection reagent is a cationic lipid transfection reagent.

13. The method of claim 1, wherein the mRNA agent comprises mRNA.

14. The method of claim 1, wherein the platelets are loaded with the mRNA agent in a period of time of 1 minute to 48 hours.

15. The method of claim 1, wherein the concentration of mRNA agent in the mRNA agent-loaded platelets is from about 0.1 nM to about 10 μM.

16. The method of claim 1, wherein the one or more organic solvents selected from the group consisting of ethanol, acetic acid, acetone, acetonitrile, dimethylformamide, dimethyl sulfoxide, dioxane, methanol, n-propanol, isopropanol, tetrahydrofuran (THF), N-methyl pyrrolidone, dimethylacetamide (DMAC), and combinations thereof.

17. The method of claim 1, further comprising cold storing, cryopreserving, freeze-drying, thawing, rehydrating, or combinations thereof the mRNA agent-loaded platelets.

18. The method of claim 17, wherein the method comprises freeze-drying the mRNA agent-loaded platelets.

19. The method of claim 18, further comprising rehydrating the mRNA agent-loaded platelets obtained from the drying step.

20. mRNA agent-loaded platelets prepared by the method of claim 1.

21. Rehydrated mRNA agent-loaded platelets prepared by a method comprising rehydrating the mRNA agent-loaded platelets of claim 20.

22. The method of claim 1, wherein the method does not comprise contacting the platelets with an organic solvent.
